(19) United States
(12) Patent Application Publication
(10) Pub. No.: US 2004/0157951 A1
(43) Pub. Date: Aug. 12, 2004

(54) Monodisperse preparations useful with implanted devices

(76) Inventor: David E. Wolf, Hudson, MA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/706,687

(22) Filed: Nov. 12, 2003

Related U.S. Application Data
(63) Continuation of application No. 09/439,545, filed on Nov. 12, 1999, now abandoned.
(60) Provisional application No. 60/108,290, filed on Nov. 13, 1998.

(51) Int. Cl. C12Q 1/68; A61F 2/00

(52) U.S. Cl. 523/113; 435/6

(57) ABSTRACT

An article is disclosed that includes a core and a selectively permeable coating enclosing the core. The coating includes a monodisperse polymer.

### CROSS-REFERENCE TO RELATED APPLICATIONS

This application claims priority to U.S. provisional application Serial No. 60/108,290, filed Nov. 13, 1998, which is incorporated herein in its entirety.

### BACKGROUND OF THE INVENTION

The invention relates to preparations including monodisperse polymers.

Available implantable devices often include a core, e.g., a gel (e.g., a hydrogel) and a permeable coating enclosing the core. The coating often has a porosity that prevents components of the implant recipient's immune system from entering the core. When the core includes a therapeutic substance, the coating aids in preventing the components of the immune system from entering the core and destroying the cells within the implantable device. Many methods for making implantable devices are known in the art.

Polyamino acids having a polydisperse molecular weight have been used to coat implantable devices. These polyamino acids are often prepared by base-initiated polymerization of the corresponding N-carboxyanhydride, and have a mixture of polymer chains differing in the degree of polymerization (i.e., molecular weight).

### SUMMARY OF THE INVENTION

In a first aspect, the invention features an article that includes a core and a selectively permeable coating enclosing the core, the coating including a monodisperse (i.e., size-homogeneous) polymer. In one embodiment, the polymer has a molecular weight of from about 1,000 to about 60,000 Da. In another embodiment, the polymer has a molecular weight of from about 1,000 to about 10,000 Da. In other embodiments, the polymer has a molecular weight of from about 1,000 to about 4,000 Da. In some embodiments, the polymer includes from about 10 to about 300 monomer units. In other embodiments, the polymer includes from about 10 to about 40 monomer units. In some embodiments, the polymer includes from about 20 to about 30 monomer units.

The polymer can be selected from the group consisting of homopolymers and heteropolymers. The polymer can also be selected from the group consisting of homopolyamino acids, heteropolyamino acids, homooligonucleotides and heterooligonucleotides. Other polymers include amino acids selected from the group consisting of D-amino acids, L-amino acids and combinations thereof.

In one embodiment, the polymer is a polyamino acid having a length of from about 10 to about 300 amino acids. the polymer is a polyamino acid having a length of from about 10 to about 30 amino acids.

In some embodiments the core includes a hydrogel. In other embodiments, the core includes a gel selected from the group consisting of alginate, agar, collagen, chitosan, gelatin, and combinations thereof. In one embodiment, the core includes a gel selected from the group consisting of polyacrylamide, polyacrylate, polymethacrylate, and combinations thereof.

In other embodiments, the article further includes a second selectively permeable coating on the first coating, the second coating including a monodisperse polymer. In one embodiment, the monodisperse polymer of the second coating is different from the monodisperse polymer of the first coating. In some embodiments, the coating further includes a second monodisperse polymer.

In a second aspect, the invention features a device that includes an article described herein, wherein the core further includes a source of a therapeutic substance. In one embodiment, the therapeutic substance includes at least one cell. Examples of useful cells include primary tissue cells, cultured cell lines, genetically engineered cells, stem cells, and combinations thereof. In one embodiment, the therapeutic substance includes an islet.

In other embodiments, the therapeutic substance includes a drug. In some embodiments, the therapeutic substance includes a component capable of a producing a drug.

In some embodiments the device further includes a second selectively permeable coating on the first selectively permeable coating, the second selectively permeable coating including a monodisperse polymer. In another embodiment, the second monodisperse polymer differs from the first monodisperse polymer. In one embodiment, the device further includes a gel matrix, and an article described herein is disposed in the gel matrix. In another embodiment, the device further includes a second selectively permeable polymer (e.g., a monodisperse polymer) coating enclosing the gel matrix.

The device can further include a second gel matrix surrounding the second selectively permeable polymer coating. In one embodiment, the gel matrix is biocompatible. In other embodiments, the second selectively permeable polymer coating is immunoisolating. In some embodiments, the device further includes a gel matrix and a plurality of the articles disposed in the gel matrix. In one embodiment, the device further includes a second selectively permeable polymer coating enclosing the gel matrix.

In a third aspect, the invention features a device that includes an article described herein and an agent for detecting an analyte disposed in the core of the article. In one embodiment, the device further includes a gel matrix, and the article is disposed in the gel matrix. The device can further include a second selectively permeable polymer coating enclosing the gel matrix. In some embodiments, the device further includes a second gel matrix surrounding the second selectively permeable polymer coating. Preferably the gel matrix is biocompatible and the coating is immunoisolating.

In one embodiment, the agent is capable of binding to the analyte. In other embodiments, the agent is capable of reversibly binding to the analyte. In some embodiments, the agent is capable of reacting with the analyte. Useful agents include a reagent selected from the group consisting of energy absorbing reagents, spin resonance reagents, x-ray reagents and combinations thereof.

Preferably the agent includes a fluorescence reagent. In one embodiment, the fluorescence reagent exhibits a fluorescence intensity, an emission spectrum, an excitation spectrum, or an excited state lifetime in the presence of the analyte that is different from its fluorescence intensity, emission spectrum, excitation spectrum, or excited state lifetime in the absence of the analyte.

In other embodiments, the fluorescence reagent includes an energy-accepting donor molecule and an energy-absorbing acceptor molecule, the excited state energy level of the donor overlapping with the excited state energy level of the acceptor.

In one embodiment, the reagent is retained within the device while the analyte is allowed to diffuse into and out of the device.

In other embodiments, the device is implantable in an individual.

In a fourth aspect, the invention features an in vivo method for determining an analyte in the body fluids of an individual comprising the steps of

a) placing a device as described herein in communication with the body fluids of the individual suspected of containing the analyte in such a way that once in place the device does not exit the skin of the individual,

the agent comprising a fluorescence reagent for detecting the analyte that reversibly binds to the analyte,

the fluorescence reagent having a fluorescence intensity, an emission spectrum, an excitation spectrum, or an excited state lifetime in the presence of the analyte that is different from its fluorescence intensity, emission spectrum, excitation spectrum, or excited state lifetime in the absence of the analyte,

the device being configured to retain the fluorescence reagent while allowing the analyte to diffuse into and out of the device;

b) transdermally illuminating the device; and

c) measuring the fluorescence intensity, emission spectrum, excitation spectrum, or excited state lifetime of the fluorescence reagent relative to the fluorescence intensity, emission spectrum, excitation spectrum, or excited state lifetime of the fluorescence reagent in the absence of the analyte; and

d) correlating the change in fluorescence intensity, emission spectrum, excitation spectrum, or excited state lifetime of the fluorescence reagent with the presence or amount of the analyte in the individual.

In one embodiment, the analyte is a carbohydrate. In another embodiment, the carbohydrate is glucose or a derivative thereof.

In a fifth aspect, the invention features an in vivo method for determining an analyte in the body fluids of an individual comprising the steps of

a) placing the device as described herein in communication with the body fluids of the individual suspected of containing the analyte,

the agent comprising a fluorescence reagent for detecting the analyte that reversibly binds to the analyte,

the device being configured to retain the fluorescence reagent while allowing analyte to diffuse into and out of the device,

the fluorescence reagent comprising an energy-absorbing donor molecule and an energy-absorbing acceptor molecule, the excited state energy level of the donor overlapping with the excited state energy level of the acceptor;

b) transdermally illuminating the device so as to

i) excite the donor or

ii) excite both the donor and acceptor; and

c) measuring the fluorescence from the fluorescence reagent associated with the presence of the analyte in the individual by determining the extent to which non-radiative fluorescence resonance energy transfer occurs between the donor and the acceptor upon binding,

the non-radiative fluorescence resonance energy transfer being determined by measuring

i) the ratio of the fluorescence signal at two emission wavelengths, one of which is due to donor emission and the other of which is due to acceptor emission, when only the donor is excited,

ii) the ratio of the fluorescence signal due to the acceptor following donor excitation and the fluorescence signal due to the acceptor following acceptor excitation,

iii) a change in donor lifetime,

iv) quenching of donor fluorescence, or

v) an enhancement of acceptor fluorescence intensity; and

d) correlating the non-radiative fluorescence resonance energy transfer with the presence or amount of the analyte in the individual.

In a sixth aspect, the invention features a method of making an article, where the method includes applying a selectively permeable monodisperse polymer coating on a core.

In a seventh aspect, the invention features, a monodisperse preparation of a polyamino acid. In preferred embodiments, the polyamino acid has a molecular weight of about 1,000, 2000, 3,000, 4,000, 5,000, 7,000, 8,000, 10,000, 15,000, 30,000, 40,000, 50,000 or 60,000 Da. In preferred embodiments, the polyamino acid has a molecular weight of between 1,500-3,000; 1,500-5,000; 1,500-10,000; 3,000-10,000; or 10,000-15,000 Da. In preferred embodiments, the polyamino acid has a length of about 10, 20, 30, 40 50, 60, 70, 80, 90, 100, 120, 140, 180, 200, 230, 260 Da or 300 amino acids. In preferred embodiments, the polyamino has a length of between 50-75, 50-100, 50-200, or 50-500 amino acids.

In preferred embodiments, the polyamino acid is comprised of residues having a basic side chain; the polyamino acid is selected from the group consisting of polylysine or polyornithine. In preferred embodiments, the preparation includes at least about 0.1 mg, 0.5 mg, 1.0 mg, 5.0 mg, 10.0 mg. 50.0 mg, 100.0 mg, 500.0 mg, 750.0 mg, 1.0 g, 5.0 g, 10.0 g, 50.0 g, 100 g, or 500 g of polyamino acid.

In an eighth aspect, the invention features, a selectively permeable coating which includes or is made from a monodisperse preparation of a polyamino acid.

In a ninth aspect, the invention features, an implantable device which includes a selectively permeable coating of a size homogeneous preparation of a polyamino acid.

In preferred embodiments, the device includes a source of a therapeutic substance, e.g., a cell, disposed within the coating.

In preferred embodiments, the device includes a diagnostic biosensor disposed within the coating.

In preferred embodiments, the device is a microcapsule comprising a shape retaining gel, e.g., an alginate matrix.

In a tenth aspect, the invention features, a method for preparing a monodisperse preparation of a polyamino acid. The method includes: synthesizing a first polyamino acid polymer of a defined length; synthesizing a second polyamino acid polymer of a defined length combining the first and second polyamino acid polymers to form a monodisperse preparation. In preferred embodiments, the first and second polyamino acid are substantially identical.

In preferred embodiments, the method further includes the step of coupling, e.g., a chemical bond is formed, e.g., a covalent bond, at least two polyamino acids of defined length together. In preferred embodiments, the synthesizing is conducted by a solid phase peptide synthesis procedure.

In an eleventh aspect, the invention features, a process for preparing a selectively permeable coating composition which includes a monodisperse polyamino acid. The method includes: synthesizing polyamino acid polymers of defined length; contacting the defined length polymers with a carrier, diluent, or solvent, thereby forming a selectively permeable coating composition. In preferred embodiments, at least two polyamino acid polymers, e.g., two substantially identical polyamino acid polymers, are combined to form the preparation,

In preferred embodiments, the method further includes the step of coupling, e.g., a chemical bond is formed, e.g., a covalent bond, at least two polyamino acids of defined length together. In preferred embodiments, the synthesizing is conducted by a solid phase peptide synthesis procedure.

In a twelfth aspect, the invention features, a method of making an implantable device having an immunoisolated component. The method includes: providing said component, enclosing said component in an enclosure, wherein, at least a portion of the enclosure is formed by a layer of a monodisperse polyamino acid.

In preferred embodiments, the device includes a source of a therapeutic substance, e.g., a cell, disposed within the enclosure. In preferred embodiments, the device includes a diagnostic biosensor disposed within the enclosure. In preferred embodiments, the device is a microcapsule comprising a shape retaining gel, e.g., an alginate matrix.

A variety of therapeutic devices can be used to contain a source of a therapeutic substance, often cells, which source provides the substance to a host or recipient subject. Such devices include implantable devices, of both the diffusion and perfusion types, and extra corporeal devices, e.g., those through which blood of the host or recipient is passed. In such devices, host molecules can attack the source of the therapeutic substance and impair the function of the device. Selectively permeable components are used to inhibit the ability of host molecules to enter the device and attack the source of therapeutic substance.

Accordingly, the invention features, an implantable device which includes a sensor or a source of a therapeutic substance, e.g., an islet, disposed within a selectively permeable enclosure, all or part of which is a coating or layer of a monodisperse polyamino acid.

In preferred embodiments, the implantable device includes a cell or tissue. The cell or tissue can be autologous, allogeneic, or xenogeneic, with regard to the subject. A xenogeneic cell or tissue can be from a species which is concordant or discordant with the subject. The cell or tissue can be from the subject, but if it is from the subject, it is preferably genetically engineered to express a substance not normally expressed by or on that cell or tissue. In preferred embodiments, the cell or tissue is from a dog, pig, goat, rabbit, horse, cow, sheep, or a non-human primate species. In preferred embodiments, the cell is a pancreatic islet cell. In preferred embodiments, the pancreatic islet is from a dog, pig, goat, rabbit, horse, cow, sheep, or a non-human primate. In particularly preferred embodiments, the pancreatic islet is from a pig. In preferred embodiments, the pancreatic islet is from a human other than the subject.

In preferred embodiments, the cell or tissue is genetically engineered.

The cell or tissue can be from the pancreas, adrenal gland, brain, kidney, liver, thymus, parathyroid or thyroid. In a preferred embodiment, the cell is a cultured cell. In a preferred embodiment, the cell is from a primary culture. In a preferred embodiment, the cell has been treated with a cytokine or a growth factor.

In preferred embodiments, the cell is an immortalized cell; the cell is a blood cell; the cell or tissue is fetal; the cell is a skin, astroglial, or myoblast cell.

In preferred embodiments, the source of a therapeutic substance (and preferably the capture agent) is immunoisolated from the host, e.g., it is isolated from contact with one or more host immune components, e.g., antibodies or components of the complement system.

In preferred embodiments, the implantable device is a perfusion device, e.g., devices through which the flow of blood is directed, e.g., intravascular devices, as e.g., in an arterial or venous shunt.

In preferred embodiments, the device is a non-perfusion or passive diffusion device, e.g., a microcapsule or a macrocapsular device, e.g., a hollow fiber, a membrane chamber, or other device which separates the source of a therapeutic substance (and preferably the capture agent) from the host by an artificial semi-permeable barrier.

In preferred embodiments, the device serves to physically contain the source of a therapeutic substance, e.g., donor cells or tissues, (and preferably the capture agent), keeping them in a contained location, at least temporarily separated from the implantation site or tissues of the host.

In preferred embodiments, the device is a microcapsule or macrocapsule. It can include a gel member, e.g., a shape-retaining gel member, in which a source of a therapeutic substance, e.g., a cell or tissue, is embedded. The gel can be a hydrogel. In preferred embodiments, the hydrogel includes agarose, agar, collagen, polyethylene glycol (PEG), polyethylene oxide (PEO), or alginate. The agarose or alginate can have a high number of guluronic acid or a high number of mannuronic acid monomers. The microcapsule or macrocapsule includes a selectively permeable membrane or coating of a monodisperse polyamino acid which surrounds a gel component, e.g. a gel core in which a cell or tissue is embedded.

In a thirteenth aspect, the invention features, a composite microreactor which includes:

(a) one, or a plurality, of an internal particle which includes:

(i) a source of a therapeutic substance, e.g., an islet;

(ii) an internal particle matrix, e.g., a gel core or a solid particle, which contacts the source,

(iii) (optionally) an internal selectively permeable particle coating of a monodisperse polyamino acid enclosing the internal particle matrix; and

(b) a super matrix, e.g., a gel super matrix, in which the internal particle (or particles) is embedded; and

(c) (optionally) an outer selectively permeable coating of a monodisperse polyamino acid enclosing the super matrix,

the composite microreactor preferably providing a molecular weight cutoff that prevents molecules larger than about 400,000 daltons from coming into contact with the source.

In a fourteenth aspect, the invention features, a double composite microreactor which includes:

(1) one, or a plurality, of an internal particle which includes:

(a) a source of a therapeutic substance, e.g., an islet;

(b) an internal particle matrix which contacts the source; and

(c) (optionally) an internal particle selectively permeable coating of a monodisperse polyamino acid enclosing the first internal particle matrix;

(2) one, or a plurality, of a particle which includes:

(a) the internal particle or particles of (1)

(b) a particle matrix in which the internal particle (or internal particles) is embedded; and

(c) (optionally) a particle selectively permeable coating of a monodisperse polyamino acid enclosing the particle;

(3) a super matrix in which the particle (or particles) of (2) is embedded; and

(4) (optionally) a super matrix or outer selectively permeable coating, e.g., of polylysine enclosing the super matrix.

In preferred embodiments: an internal particle is coated with the three-part composite layer described herein; a particle is coated with the three-part composite layer described herein; an internal particle and a particle are coated with the three-part composite layer described herein; an internal particle does not include the three-part layer but a particle is coated with the three-part composite layer described herein.

In a fifteenth aspect, the invention features, a composite microreactor which includes:

(a) one, or a plurality, of an internal particle which includes:

(i) pig islet cells as a source of a therapeutic substance;

(ii) an internal particle matrix of alginate;

(iii) an internal selectively permeable particle coating of monodisperse low molecular weight polylysine enclosing the internal particle matrix; and

(b) a super matrix of alginate in which the internal particle (or particles) is embedded; and

(c) an outer selectively permeable coating of monodisperse polylysine, e.g., low molecular weight polylysine, enclosing the super matrix,

the composite microreactor preferably providing a molecular weight cutoff that prevents molecules larger than about 400,000 daltons from coming into contact with the source and having an antibody which reacts with a swine antigen, e.g., a SLA class I or class II antigen, as a capture agent attached to the outer coating of polylysine.

In a sixteenth aspect, the invention features providing a subject with a therapeutic substance. The method includes implanting in the subject an implantable device which includes a source of a therapeutic substance and a capture agent, e.g., an implantable device described herein.

In a seventeenth aspect, the invention features an implantable or extracorporeal device through which is passed a host fluid, e.g., blood. (After passage through the device the host fluid is returned to the host.) The device includes a source of a therapeutic substance, e.g., an islet. The source is separated from the host body fluid by a selectively permeable component formed of a monodisperse polyamino acid.

In preferred embodiments, the device includes a cell or tissue. The cell or tissue can be autologous, allogeneic, or xenogeneic, with regard to the subject. A xenogeneic cell or tissue can be from a species which is concordant or discordant with the subject. The cell or tissue can be from the subject, but if it is from the subject, it is preferably genetically engineered to express a substance not normally expressed by or on that cell or tissue.

In preferred embodiments, the cell or tissue is from a dog, pig, goat, rabbit, horse, cow, or sheep, or a non-human primate species. In preferred embodiments, the cell is a pancreatic islet cell. In preferred embodiments, the pancreatic islet is from a dog, pig, goat, rabbit, horse, cow, sheep, or a non-human primate. In particularly preferred embodiments, the pancreatic islet is from a pig. In preferred embodiments, the pancreatic islet is from a human other than the subject.

In preferred embodiments, the cell or tissue is genetically engineered.

The cell or tissue can be from the pancreas, adrenal gland, brain, kidney, liver, thymus, parathyroid or thyroid. In a preferred embodiment, the cell is a cultured cell. In a preferred embodiment, the cell is from a primary culture. In a preferred embodiment, the cell has been treated with a cytokine or a growth factor.

In preferred embodiments: the cell is an immortalized cell; the cell is a blood cell; the cell or tissue is fetal; the cell is a skin, astroglial, or myoblast cell.

In preferred embodiments, the device includes a port for admitting flow of the body fluid into the device which port communicates with a chamber which encloses a source of a therapeutic substance, e.g., an islet, and a capture agent, e.g., an antibody which binds antigens or epitopes other than the therapeutic substance released by the source. The source is separated from the host fluid by a selectively permeable component, and preferably, the capture agent is separated from the source by a selectively permeable component. The fluid exits the device by the same port or by a second port. The device can be used in “batch” or continuous flow fashion.

In preferred embodiments the selectively permeable component includes the three-part composite layer described herein.

The methods of the invention allow implanting of allogeneic or xenogeneic tissue with little or no immunosuppression.

The terms “monodisperse polymer” and “size-homogeneous polymer” refer to a polymer having a specific molecular weight.

The term “alginate” includes alginate derivatives.

Selectively permeable coatings of polyamino acids made with monodisperse polyamino acid polymer preparations have advantageous properties. The selectively permeable monodisperse polymer coatings provide selectively permeable membranes on particles such as gels. The polymer coatings can be selective as to molecular weight or charge and can be selected to prevent the passage of some molecules across the membrane. Some molecules are prevented from passing into the core of the article and some components within the core, e.g., gel, of the article are prevented from passing out of the article.

The articles can be constructed to be biocompatible, immunoisolating and combinations thereof.

Unless otherwise defined, all technical and scientific terms used herein have the same meaning as commonly understood by one of ordinary skill in the art to which this invention belongs. Although methods and materials similar or equivalent to those herein can be used in the practice or testing of the present invention, the preferred methods and materials are described below. All publications, patent applications, patents, and other references mentioned herein are incorporated by reference. In addition, the materials, methods, and examples are illustrative only and not intended to be limiting.

Other features and advantages of the invention will be apparent from the following detailed description and from the claims.

### BRIEF DESCRIPTION OF THE DRAWINGS

FIG. 1A is a graphic representation of absorbance and emission spectra of donor and acceptor moleuces.

FIG. 1B is a representation of non-radiative energy transfer.

FIG. 2 is a schematic diagram of a composite microreactor.

FIG. 3 is a schematic diagram of a double composite microreactor.

FIG. 4 is a plot of the leakage of Cy3.5-HSA fluorescent component from microsphere beads coated with 33, 47, and 60 peptide residue monodisperse PLL, over time.

FIG. 5 is a plot of the leakage of Cy5.5-ConA fluorescent component from the microsphere beads coated with three types of monodisperse PLL, over time.

FIG. 6 is the fluorescence spectra of the Cy3.5-HSA and Cy5.5-ConA components of sensors before and after the addition of 500 mg/dL of glucose to the sensors as measured by a spectrofluorimeter.

FIG. 7 is the kinetic response of the sensors of FIG. 6 to 500 mg/dL glucose.

### DETAILED DESCRIPTION

The article includes a core and a selectively permeable coating, which includes a monodisperse polymer, enclosing the core. The article is preferably implantable in an individual.

Useful monodisperse polymers include heteropolymers and homopolymers. Examples of useful polymers include polyamino acids, e.g., heteropolyamino acids and homopolyamino acids, polynucleotides, e.g., heterooligonucleotides and homooligonucleotides, and combinations thereof. Particularly useful polyamino acids include polylysine and polyornithine.

Preferably the polymer has a molecular weight of from about 1,000 to about 60,000 Da, more preferably from about 1,000 to about 10,000 Da, most preferably from about 1,000 to about 4,000 Da. Useful polymers have molecular weights of about 1,000, 2000, 3,000, 4,000, 5,000, 7,000, 8,000, 10,000, 15,000, 30,000, 40,000, 50,000 or 60,000 Da.

The polymer preferably includes from about 10 to about 300 monomer units, more preferably from about 10 to about 40 monomer units, more preferably from about 20 to 30 monomer units.

Preferably the polymer is a polyamino acid, e.g., polylysine, having a length of from about 10 to about 300 amino acids, more preferably from about 10 to 40 amino acids, most preferably from about 20 about 30 amino acids. Suitable polyamino acids have lengths of about 10, 20, 30, 40 50, 60, 70, 80, 90, 100, 120, 140, 180, 200, 230, 260 or 300 amino acids. Other useful polyamino acids have a length of between 20-500, 50-75, 50-100, 50-200, or 50-500 amino acids. Useful commercially available monodisperse polyamino acids include poly-L-lysine monodisperse homopolymers having 33, 47 or 60 residues available from Sigma Chemical Co. St. Louis, Mo. Other suitable monodisperse polyamino acids are available from Boehringer Mannheim (Germany).

One example of a useful method of making a monodisperse polyamino acid includes solid phase peptide synthesis.

The core can include a variety of components including, e.g., an aqueous solution, or a gel (e.g., a hydrogel). Preferably the core includes a gel. The gel preferably is capable of forming microbeads having good mechanical strength. Examples of useful gels include hydrogels, e.g., alginate, carrageenan, gum, e.g., xanthan gum, agarose, agar, collagen, gelatin, chitosan, polyethylene glycol (PEG), polyethylene oxide (PEO), and combinations thereof. Other useful gels include polyacrylamide, polyacrylate, polymethacrylate and combinations thereof.

Preferably the gel includes alginate. Alginate is particularly useful because it allows rapid polymerization and immobilization of cells at room temperature using relatively benign CaC₁₂, provides consistent gel rheology that can be conveniently varied by increasing the alginate concentration, and produces microbeads with good mechanical strength.

Alginate is composed of blocks of 1,4 linked (-D-mannuronic acid (M) and (-1-guluronic acid (G) linked together, e.g., in alternating MG blocks. Preferably the alginate is formulated with a high G block content, e.g., at least about 60% G block. The higher the percentage of G blocks in the alginate composition, the greater the pore sized and the strength of the gel matrix that is obtained in the final product. Alginate gels with a high M block content appear to be more immunogenic than gels with a high G block content.

The gels can be cross-linked using a variety of methods including, e.g., by the addition of ions including calcium, potassium, barium and combinations thereof, by changing the temperature (e.g., heating), by exposure to radiation, and combinations thereof. For temperature changing methods, care should be taken to choose temperatures appropriate to the components of the article including, e.g., the viability of living cells.

For those applications in which cells are to be dispersed in the gel, the gel preferably is sufficiently viscous to maintain the cells in a dispersed state. Such gels preferably include no greater than about 3% by weight alginate, more preferably from about 1 to about 2% by weight alginate. The gel is preferably formed by crosslinking a solution to form a semisolid gel.

Suitable methods for making hydrogel microbeads include, e.g., emulsification, electrospraying, dripping and Raleigh jet. A preferred method for making hydrogel microbeads is with an air jet.

The selectively permeable monodisperse polymer coating can be selected to have a particular molecular weight cutoff. As used herein, “molecular weight cutoff” refers to the size of the largest molecule that is not substantially blocked, e.g., by a selectively permeable coating surrounding the core, by the gel matrix, itself, or a combination thereof. Molecules with a molecular weight above the cutoff are substantially prevented from entering or leaving the article.

The article preferably exhibits a molecular weight cutoff of about 400 kDa, more preferably about 150 kDa, more preferably about 100 kDa, and most preferably about 50 kDa. In preferred embodiments, the molecular weight cutoff is sufficient to prevent Ig molecules, e.g., IgG and complement, from entering the article and coming into contact with the core.

The article is preferably a microsphere. The article can include a number of layers where individual layers include, e.g., a selectively permeable monodisperse polymer coating, a gel matrix, and combinations thereof. The selectively permeable monodisperse polymer coatings can include the same or different monodisperse polymers. Examples of various article structures are described below. Preferably the coating is immunoisolating, the gel is biocompatible, and the article is immunoisolating and biocompatible.

The articles can be constructed to be suitable for a variety of applications including, e.g., in vitro, in vivo and combinations thereof.

Coating with Monodisperse Polylysine

The monodisperse polymer coating can be applied according to a variety of methods including, e.g., “electrostatic” coating.

One useful method for coating a core (e.g., an alginate core) with monodisperse polylysine includes dropping the core into a solution that includes monodisperse polylysine in a serum free culture medium. The solution includes an amount of polymer sufficient to coat the core. Useful polymer concentrations include from about 0.01% by weight polylysine to about 20% by weight polylysine, more preferably from about 0.01% to about 1.0% by weight polylysine based upon the coating composition. For coating sensors, the polylysine is preferably in a buffer solution. Preferably the solution includes 0.05% monodisperse polylysine. For microreactors, the coating solution preferably includes a culture medium. The thickness of the monodisperse polylysine coating can be increased by increasing the time the core is left in the solution, or alternatively, by increasing the concentration of the solution. Preferably the ratio of the volume of beads to the volume of solution is, e.g., 1:5, 1:10, 1:15, or 1:20. For smaller beads a greater proportion of solution is desirable.

Another suitable method includes coating a solid core and then altering the solid core. For example, a frozen, solid agarose, or a plastic core can be coated and then subsequently liquefied, e.g., heated, or contacted with a liquefying agent (e.g., a calcium chelator), e.g., by passing the agent through the selectively permeably coating so as to cause the solid, e.g., agarose or plastic, to liquefy and, optionally, exit (diffuse out of) the membrane.

Implantable Devices

Implantable Biosensors

Implantable biosensors, e.g., such as those described in U.S. Pat. No. 5,342,789 and U.S. Ser. No. 08/467,915, entitled, “Method and Device for Detecting and Quantifying Substances in Body Fluids,” filed Jun. 6, 1995 (both of which are incorporated herein), can be encapsulated or otherwise coated using materials or methods described herein.

The article can be a sensor capable of detecting substances in body fluids. Preferably the sensor is implantable in an individual, e.g., a mammal, e.g., a human. The core of the article includes an agent capable of detecting the presence of an analyte. The selectively permeable monodisperse polymer coating of the article is selected to allow analyte to diffuse into the core such that it contacts the agent, while maintaining the agent within the article. The selectively permeable monodisperse polymer coating is preferably selectively permeable relative to at least one analyte of interest.

The agent can include more than one component. Examples of useful agents include energy absorbing, e.g., light absorbing and sound absorbing, reagents, x-ray reagents, spin resonance reagents, nuclear magnetic resonance reagents, and combinations thereof.

Preferably the sensor uses fluorescence techniques, e.g., non-radiative fluorescence resonance energy transfer (FRET). As used herein, “detecting” may include qualitatively determining the presence of an analyte, as well as quantitatively measuring its concentration. The sensor is placed in communication with body fluids, e.g., blood, plasma, or serum, of an individual. The sensor can be placed in the individual in such a way that once in place the sensor does not exit the skin of the individual.

The sensor can include a fluorescence reagent for detecting the analyte and is configured to retain the fluorescence reagent while allowing analyte to diffuse into and out of the sensor. In this way, the sensor can be used for extended monitoring because depletion of the fluorescence reagent is minimized, if not eliminated. Once the sensor is in place, it is illuminated with radiation transdermally and the fluorescence from the fluorescence reagent associated with the presence of the analyte is measured.

As used herein, “fluorescence” refers to radiation emitted in response to excitation by radiation of a particular wavelength. It includes both short lived (nanosecond range) and long-lived excited state lifetimes; the latter is sometimes referred to as phosphorescence.

As used herein, “fluorescence reagent” refers to a material whose fluorescence behavior (e.g., intensity, emission spectrum, or excitation spectrum) changes in the presence of the analyte being detected.

In some embodiments, the reagent binds reversibly to the analyte. For example, the reagent may be a fluorophore (or compound labeled with such a molecule) that binds directly to the analyte. It is the fluorescence behavior of this molecule (or compound labeled with this molecule) that changes as a result of analyte binding.

The reagent may also include more than one component. For example, it may include an analogue to the analyte labeled with a fluorophore and a ligand (e.g., an antibody, receptor for the analyte, lectin, enzyme, or lipoprotein) that binds competitively (and specifically) to the analogue and the analyte. In this case, it is the fluorescence behavior of the labeled analogue that changes as a result of ligand binding to analyte. Conversely, ligand may be labeled, rather than the analogue, in which case it is the fluorescence behavior of the labeled ligand that changes.

The reagent may also include two components, one of which is labeled with an energy-absorbing donor molecule and the other of which is labeled with an energy-absorbing acceptor molecule; the donor and acceptor having overlapping excited state energy levels. One or both molecules forming the donor-acceptor pair can be fluorophores. Regardless, however, it is the fluorescence associated with the FRET from donor to acceptor that is measured. The components maybe members of a specific binding pair (e.g., an analogue of the analyte and a ligand capable of binding competitively (and specifically) to both the analogue and the analyte) or ligands (e.g., antibodies or oligonucleotides) that bind specifically to different portions of the analyte.

FRET can also be measured where a single reagent capable of binding to the analyte is labeled with both donor and acceptor molecules.

As used herein, “fluorophore” refers to a molecule that absorbs energy and then emits light.

As used herein “analogue” refers to a material that has at least some binding properties in common with those of the analyte such that there are ligands that bind to both. The analogue and the analyte, however, do not bind to each other. The analogue may be a derivative of the analyte such as a compound prepared by introducing functional chemical groups onto the analyte that do not affect at least some of the binding properties of the analyte. Another example of a derivative is a lower molecular weight version of the analyte which nonetheless retains at least some of the binding properties of the analyte.

In other embodiments, the fluorescence reagent does not bind to the analyte except transiently. In other words, binding to analyte in and of itself is not the cause of the detectable fluorescence change. Instead, in these embodiments detection involves an irreversible chemical reaction, with an accompanying change in fluorescence; the chemical reaction may be proceeded by binding to analyte.

In one example, the fluorescence reagent may include an enzyme and a co-factor or substrate that reacts with the analyte in the presence of the enzyme with an accompanying fluorescence change. In the case where the enzyme reacts with substrate, a co-factor may also be present to assist the reaction.

In another example, the analyte is an enzyme and the fluorescence reagent includes a substrate that is altered (e.g., cleaved) by the enzyme with an accompanying fluorescence change. In yet another example, the fluorescence reagent includes (a) an enzyme in whose presence the analyte reacts to form a detectable product, and (b) an indicator for detecting the presence of the product.

In another example, the fluorescence reagent includes (a) a substrate or co-factor capable of reacting with an enzyme to cause a detectable fluorescence change, (b) a first ligand labeled with a first portion of that enzyme and capable of binding to a first portion of the analyte, and (c) a second ligand labeled with a second (different) portion of the analyte. Neither the first portion nor the second portion of the enzyme is capable individually of interacting with the substrate or the co-factor to produce a detectable fluorescence change. However, binding of the first and second ligands to the analyte causes the first and second enzyme portions to interact with each other, thereby reconstituting the enzyme. The reconstituted enzyme can then react with the substrate or co-factor, with a concomitant change in fluorescence.

A variation of this approach involves an analogue of the analyte labeled with a first portion of the enzyme and a ligand labeled with a second portion of the enzyme. In the absence of analyte, the analogue and analyte bind to each other, thereby reconstituting the enzyme and allowing reaction with substrate or co-factor, with accompanying fluorescence. The presence of analyte disrupts analogue-ligand binding, and thus reconstitution of the enzyme, with an accompanying decrease in fluorescence (since the individual enzyme portions cannot interact with the substrate or co-factor to cause a detectable fluorescence change).

Regardless of whether the fluorescence reagent binds to analyte, changes in fluorescence associated with the presence of the analyte may be measured in several ways. These changes include changes in the excited state lifetime of, or fluorescence intensity emitted by, the fluorophore (or compound labeled with the fluorophore). They also include changes in the excitation or emission spectrum of the fluorophore (or compound labeled with the fluorophore). Changes in the excitation or emission spectrum, in turn, may be measured by measuring (a) the appearance or disappearance of emission peaks, (b) the ratio of the signal observed at two or more emission wavelengths, (c) the appearance or disappearance of excitation peaks, or (d) the ratio of the signal observed at two or more excitation wavelengths.

In the case of FRET, the illumination wavelength may be selected such that it predominantly excites only the donor molecule. We say “predominantly” because due to bleed-through phenomena, it is possible that there will be some acceptor excitation as well.

Thus, as used herein, “excitation” of donor or acceptor will refer to an excitation wavelength that predominantly excites donor or acceptor.

Following excitation, non-radiative fluorescence resonance energy transfer is determined by measuring the ratio of the fluorescence signal at tow emission wavelengths, one of which is due to donor emission and the other of which is due to acceptor emission. Just as in the case of excitation, there may be some “bleeding” of the fluorescence signal such that acceptor emission makes a minor contribution to the donor emission signal, and vice versa. Thus, whenever we refer to a signal as being “due to” donor emission or acceptor emission, we mean that the signal is predominantly due to donor emission or acceptor emission.

Alternatively, the illumination may be selected such that it excites the donor at a first wavelength and the acceptor at a second wavelength. In otherwords, two separate excitation events, each at different wavelength, are used. In this case, non-radiative fluorescence energy transfer is determined by measuring the ratio of the fluorescence signal due to the acceptor following donor excitation and the fluorescence signal due to the acceptor following acceptor excitation.

FRET can also be measured by assessing whether there is a decrease in donor lifetime, a quenching of donor fluorescence intensity, or an enhancement of acceptor fluorescence intensity; the latter two are measured at a wavelength in response to excitation at a different wavelength (as opposed to the ratio measurements described above, which involve either measuring the ratio of emissions at two separate wavelengths or measuring the ratio of emission at a wavelength due to excitation at two separate wavelengths).

The sensor can be constructed to be capable of detecting a number of analytes. Suitable analytes include, for example, carbohydrates (e.g., glucose, fructose, and derivatives thereof). As used herein, “carbohydrate” refers to any of the group of organic compounds composed of carbon, hydrogen, and oxygen, including sugars, starches and celluloses. Other suitable analytes include glycoproteins (e.g., glycohemoglobin, thyroglobulin, glycosylated albumin, glycosylated albumin, and glycosylated apolipoprotein), glycopeptides, and glycolipids (e.g. sphingomyelin and the ganglioside G_{M2}).

Another group of suitable analytes includes ions. These ions may be inorganic or organic. Examples include calcium, sodium, chlorine, magnesium, potassium, bicarbonate, phosphate, and carbonate. The sensor is also useful for detecting proteins and peptides (the latter being lower molecular weight versions of the former); a number of physiological states are known to alter the level of expression of proteins in blood and other body fluids. Included in this group are enzymes (e.g., enzymes associated with cellular death such as LDH, SGOT, SGTT, and acid and alkaline phosphatases), hormones associated with pregnancy such as human chorionic gonadotropin), lipoproteins (e.g., high density, low density, and very low density lipoprotein), and antibodies (e.g., antibodies to autoimmune diseases such as AIDS, myasthenia gravis, and lupus). Antigens and haptens are also suitable analytes.

Additionally, the sensor can detect analytes such as steroids (e.g., cholesterol, estrogen, and derivatives thereof). The sensor is also useful for detecting and monitoring substances such as theophylline and creatinine.

The sensor may also be used to detect and monitor pesticides and drugs. As used herein, “drug” refers to a material that, when ingested, inhaled, absorbed or otherwise incorporated into the body produces a physiological response. Included in this group are alcohol, therapeutic drugs (e.g., chemotherapeutic agents such as cyclophosphamide, doxorubicin, vincristine, etoposide, cisplatin, and carboplatin), narcotics (e.g., cocaine and heroin) and psychoactive drugs (e.g., LSD).

The sensor may also be used to detect and monitor polynucleotides (e.g., DNA and RNA). For example, overall DNA levels may be assayed as a measure of cell lysis. Alternatively, the sensor can be used to assay for expression of specific sequences (e.g., HIV RNA).

Dual-Label Techniques and FRET

Basic Elements of FRET

FRET generally involves the non-radiative transfer of energy between two fluorophores, one an energy donor (D) and the other an energy acceptor (A). Any appropriately selected donor-acceptor pair can be used, provided that the emission of the donor overlaps with the excitation spectra of the acceptor and both members can absorb light energy at one wavelength and emit light energy of a different wavelength.

One example of a sensor is described below with particular reference to fluorescein and rhodamine as the donor-acceptor pair. As used herein, the term fluorescein refers to a class of compounds which includes a variety of related compounds and their derivatives. Similarly, as used herein, the term rhodamine refers to a class of compounds which includes a variety of related compounds and their derivatives. Other examples of donor/acceptor pairs are NBD N-(7-mitrobenz-2-oxa-1,3-diazol-4-yl) to rhodamine, NBD or fluorescein to eosin or erythrosin, dansyl to rhodamine, acridine orange to rhodamine.

Alternatively, both the donor and acceptor can absorb light energy, but only one of them emits light energy. For example, one molecule (the donor) can be fluorescent and the other (the acceptor) can be nonfluorescent. It is also possible to make use of a donor-acceptor pair in which the acceptor is not normally excited at the wavelength used to excite the (fluorescent) donor; however, nonradiative FRET causes acceptor excitation.

Although the donor and the acceptor are referred to herein as a “pair”, the two “members” of the pair can, in fact, be the same substance. Generally, the two members will be different (e.g., fluorescein and rhodamine). It is possible for one molecule (e.g., fluorescein, rhodamine) to serve as both donor and acceptor; in this case, energy transfer is determined by measuring depolarization of fluorescence.

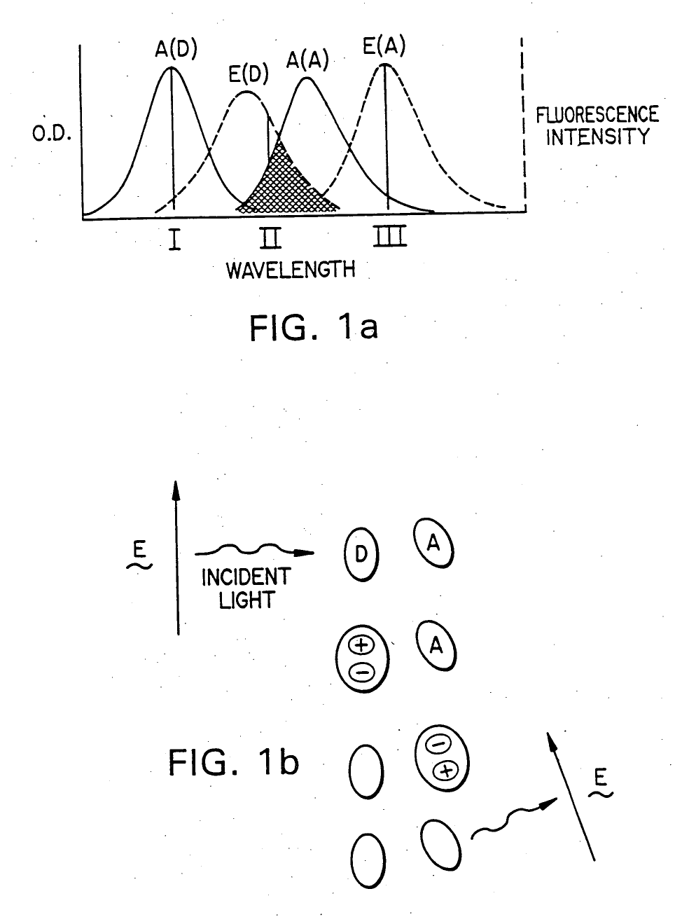

The concept of FRET is represented in FIG. 1. The absorbance and emission of donor, designated A(D), and E(D), respectively, and the absorbance and emission of acceptor, designated A(A) and E(A), respectively, are represented graphically in FIG. 1A. The area of overlap between the donor emission and the acceptor absorbance spectra (which is the overlap integral) is of importance. If excitation occurs at wavelength I, light will be emitted at wavelength II by the donor, but not at wavelength III by the acceptor because the acceptor does not absorb light at wavelength I.

The non-radiative transfer process which occurs is represented in FIG. 1B. D molecule absorbs the photon whose electric field vector is represented by E. The excited state of D is shown as a dipole with positive charge on one side and negative charge on the other. If an acceptor molecule (A) is sufficiently close to D (e.g., typically less than 100 Angstroms), an oppositely charged dipole is induced on it (it is raised to an excited state). This dipole-induced dipole interaction falls off inversely as the sixth power of donor-acceptor intermolecular distance.

Classically, partial energy transfer can occur. However, this is not what occurs in FRET, which is an all or nothing quantum mechanical event. That is, a donor is not able to give part of its energy to an acceptor. All of the energy must be transferred and energy transfer can occur only if the energy levels (i.e., the spectra) overlap. When A leaves its excited state, the emitted light is rotated or depolarized with respect to the incident light. As a result, FRET manifests itself as a decrease in fluorescence intensity (i.e., decrease in donor emission) at II, an appearance of fluorescence intensity at III (i.e., an increase in sensitized emission) and a depolarization of the fluorescence relative to the incident light.

A final manifestation of FRET is in the excited state lifetime. Fluorescence can be seen as an equilibrium process, in which the length of time a molecule remains in its excited state is a result of competition between the rate at which it is being driven into this state by the incident light and the sum of the rates driving it out of this state (fluorescence and nonradiative processes). If a further nonradiative process, FRET, is added (leaving all else unchanged), decay is favored, which means donor lifetime at II is shortened.

When two fluorophores whose excitation and emission spectra overlap are in sufficiently close proximity, the excited state energy of the donor molecule is transferred by a resonance dipole-induced dipole interaction to the neighboring acceptor fluorophore. In FRET, a sample or mixture is illuminated at a wavelength which excites the donor but not the acceptor molecule directly. The sample is then monitored at two wavelengths: that of donor emissions and that of acceptor emissions. If donor and acceptor are not in sufficiently close proximity, FRET does not occur and emissions occur only at the donor wavelength. If donor and acceptor are in sufficiently close proximity, FRET occurs. The results of this interaction are a decrease in donor lifetime, a quenching of donor fluorescence, an enhancement of acceptor fluorescence intensity, and depolarization of fluorescence intensity. The efficiency of energy transfer, Eₜ, falls off rapidly as the distance between donor and acceptor molecule, R, increases. For an isolated donor acceptor pair, the efficiency of energy transfer is expressed as:

Eₜ=1/[1+(R/Rₒ)⁶] (1)

where R is the separation distance between donor and acceptor and Rₒ is the distance for half transfer. Rₒ is a value that depends upon the overlap integral of the donor emission spectrum and the acceptor excitation spectrum, the index of refraction, the quantum yield of the donor, and the orientation of the donor emission and the acceptor absorbance moments. Forster, T., Z Naturforsch. 4A, 321-327 (1949); Forster, T., Disc. Faraday Soc. 27, 7-17 (1959).

Because of its 1/R⁶ dependence, FRET is extremely dependent on molecular distances and has been dubbed “the spectroscopic ruler”. (Stryer, L., and Haugland, R. P., Proc. Natl. Acad. Sci. USA, 98:719 (1967). For example, the technique has been useful in determining the distances between donors and acceptors for both intrinsic and extrinsic fluorophores in a variety of polymers including proteins and nucleic acids. Cardullo et al. demonstrated that the hybridization of two oligodeoxynucleotides could be monitored using FRET (Cardullo, R., et al., Proc. Natl. Acad. Sci., 85:8790-8794 (1988)).

Concept of Using FRET for Analyte Detection

In general, FRET is used for analyte detection in one of two ways. The first is a competitive assay in which an analogue to the analyte being detected and a ligand capable of binding to both analogue and analyte are labeled, one with a donor fluorophore and the other with an acceptor fluorophore. Thus, the analogue may be labeled with donor and the ligand with acceptor, or the analogue may be labeled with acceptor and the ligand with donor. When the labeled reagents contact analyte, analyte displaces analogue bound to ligand. Because ligand and analogue are no longer close enough to each other for FRET to occur, the fluorescence signal due to FRET decreases; the decrease correlates with the concentration of analyte (the correlation can be established in a prior calibration step).

To be able to reuse the fluorescence reagents, the binding between analyte and ligand should be reversible under physiological conditions. Similarly, the equilibrium binding constants associated with analyte-ligand binding and analogue-ligand binding should be such that analyte can displace analogue. In other words, analogue-ligand binding should not be so strong that analyte cannot displace analogue.

This approach is applicable to detecting carbohydrates, steroids, proteins, peptides, steroids, antigens, haptens, drugs, pesticides, theophylline, creatinin, and small organic molecules generally. In the case of carbohydrates such as glucose and fructose, suitable analogue-ligand combinations satisfying the above-described selection criteria include the following combinations: glycoconjugate-lectin, antibody-antigen, receptor-ligand, and enzyme-substrate. For example, in the case of glucose the combination of bovine serum albumin covalently labeled with glucose or glucose analogue (as the glycoconjugate) and Concanavalin A (as the lectin) have been found to be effective. To determine suitable combinations for other sugars, one can select a lectin that binds to the sugar and then use that lectin in combination with bovine serum albumin covalently labeled with that sugar or an analogous sugar.

In the case of analytes such as steroids, proteins, and peptides, the appropriate combination would be an analogue to the steroid, protein, or peptide, and an antibody (or antigen, where the protein or peptide is an antibody) or a receptor for the steroid, protein, or peptide. For example, in the case of steroids we refer to Haugland, R. P. (1989) Molecular Probes: Handbook of Fluorescent Probes and Research Chemicals, Molecular Probes, Eugene, Oreg. for preparation of suitable analogues. Using cholesterol as a representative example, derivatives can be prepared either by covalent attachment of a fluorophore (e.g., NBD or pyrene) to the aliphatic side chain or to a hydroxyl group (e.g., using anthracene as the fluorophore). For cholesterol, the molecules thus produced are respectively, 22-(N-(7-nitrobenz-2-oxa-1,3-diazol-4-yl)amino-23,24-bisnor-5-cholen-3B-ol; 1-pyrenemethyl 3B-hydroxy-22,23-bisnor-5-cholenate; and cholesteryl anthracene-9-carboxylate. The steroid can also be conjugated to a carrier protein or other macromolecule that would also be fluorescently tagged with donor or acceptor. The conjugation would again proceed via either the aliphatic side chain or the hydroxyl group.

Similar considerations apply in the case of glycoproteins, glycopeptides and glycolipids. In the case of glycosylated hemoglobin, FRET between a labeled lectin and the heme itself could be measured (this would manifest itself as a quenching of fluorescence).

The second approach using FRET is to select two ligands that bind to different portions (sites) of the analyte molecule; in addition to being spatially different, the portions may be chemically different as well. This approach is applicable to detection f antigens, haptens, steroids, proteins, peptides, drugs, pesticides, theophylline, creatinin, and large organic molecules generally. The ligands could be two antibodies, two cell receptors, or an antibody and a cell receptor. In the case of hormones such as HCG, FSH, and LSH, for example, the labeled ligands could be antibodies or cell receptors that bind to different portions of the hormone molecule.

One variation of this second approach is to detect antibodies such as anti-DNA antibodies in lupus patients by encapsulating two fluorescent DNA fragments, one labeled with donor and the other with acceptor, and then measuring FRET, which would occur if the antibody of interest were present and crosslinked the labeled fragments.

Another variation involves labeled oligonucleotide probes. As described in Cardullo, R. et al. Proc. Natl. Acad. Sci., 85:8790-8794 (1988), the hybridization of two oligodeoxynucleotides can be monitored using FRET in conjunction with such probes. In this way, specific DNA sequences can be determined.

To assay overall DNA levels, reagents that bind non-specifically to DNA or RNA are used. Examples of such reagents include fluorescent intercalating dyes that show dramatic spectral shifts upon binding.

In yet another variation, a single material is labeled with both donor and acceptor fluorophores. The fluorescence change associated with the conformational change in the material upon binding to analyte is used as an indication of analyte presence. For example, the analyte may be a helical DNA molecule and the fluorescence reagent is a material labeled with donor and acceptor fluorophores that bind to DNA. Biding changes the separation distance between the donor and acceptor, and thus the signal detected by FRET.

Concept of Using FRET to Measure Glucose Concentrations

The concept of using FRET to measure glucose concentrations in solution is set forth in U.S. Pat. No. 5,342,789, which is incorporated herein.

Single Label Technique

Other useful techniques include single label techniques. These techniques generally fall into two categories: (1) those involving direct binding of fluorophore or compound labeled with the fluorophore to the analyte of interest, and (2) those involving a competitive assay in which the analyte and an analogue thereof compete for the same ligand (either the analogue or ligand being labeled with a fluorophore. Regardless of the category however, fluorescence changes caused by the presence of analyte are detected by measuring changes in fluorescence intensity or in excitation or emission spectra as described above.

Non-Binding Technique

In this type of assay, the fluorescence reagent does not bind to the analyte (i.e., binding in and of itself does not cause the detectable fluorescence change).

One example of such a sensor includes an enzyme and a co-factor or substrate that reacts with the analyte in the presence of the enzyme, with an accompanying fluorescence change. For example, the sensor may be an ethanol sensor in which NAD (the co-factor) and alcohol dehydrogenase (the enzyme) are included in the sensor. In the presence of ethanol, NAD is converted to NADH, which is fluorescent. The change in fluorescence upon production of NADH is used to detect the presence and concentration of alcohol in the body.

Another example of such a sensor is glucose sensor that proceeds by way of two reactions: an initial enzyme reaction that converts glucose into a detectable product and an indicator reaction that detects the product. In the first reaction, glucose reacts in the presence of a first enzyme (glucose oxidase) to produce hydrogen peroxide. In the second reaction, the presence of hydrogen peroxide is detected by a dye (leuco dye) in a reaction catalyzed by a second enzyme (peroxidase). The change in fluorescence upon production of hydrogen peroxide (as evidenced by the dye) indicates the presence and concentration of glucose. The sensor thus includes two enzymes (peroxidase and glucose oxidase) and a fluorophore (e.g., leuco dye) sensitive to the reaction product of the first reaction.

In another example of such a sensor, the analyte is an enzyme. The sensor includes a substrate that is cleaved by the enzyme to create by-products having different fluorescence properties compared to the uncleaved substrate. For example, the enzyme may be trypsin and the substrate may be N-CBZ-Gly-Gly-Arg-beta-naphthylamide HCL. Cleavage releases an N-CBZ-containing fragment whose fluorescence properties are different fromt eh uncleaved substrate.

In another example of such sensor, the sensor includes a co-factor or substrate that reacts with an enzyme to cause a detectable fluorescence change, and two ligands that bind to different portions of the analyte. Each ligand is labeled with a portion of the enzyme that reacts with the substrate or co-factor. The two enzyme portions are different from each other moreover, individually they are incapable of interacting with the substrate or co-factor to produce a detectable fluorescence change. However, when the two labeled ligands bind to analyte the individual enzyme portions interact with each other to reconstitute the enzyme (the enzyme portions are chose such that reconstitution does not occur, or occurs only to a minimal extent, until ligand binding to analyte occurs). Once the enzyme s reconstituted, it can now react with the substrate or co-factor.

In a variation of this protocol, an analogue of the analyte is labeled with one portion of the enzyme and a ligand capable of binding to both analogue and analyte is labeled with the other portion When analogue and ligand bind to each other, the enzyme is reconstituted and reaction with substrate or co-factor occurs. Once again, the enzyme portions are chosen such that reconstitution does not occur, or occurs only to a minimal extent, until analogue-ligand binding occurs. The presence of analyte disrupts analogue-ligand binding, and thus enzyme reconstitution. As a result, fluorescence associated with reaction between enzyme and substrate or co-factor decreases, the decrease being associated with analyte presence and concentration. Because reaction with substrate or co-factor occurs in the absence of analyte, the substrate/co-factor and enzyme should be chosen such that this reaction occurs slowly. This will minimize depletion of substrate/co-factor, thereby enabling the sensor to be used over extended periods of time.

Implantable Devices Including a Source of Therapeutic Substance

An implantable can include a matrix, e.g., gel, e.g., a hydrogel, or core in which a source of a therapeutic substance, e.g., living cells, are disposed and a selectively permeable coating enclosing the gel. The coating often has a porosity that prevents components of the implant recipient's immune system from entering and destroying the cells within the implantable device. Many methods for making implantable devices are known in the art. A few are cited below. These are cited merely as examples, and are not the only methods that can be used with the invention.

The methods disclosed herein can be used with the monodisperse polyamino acid preparations of the invention.

Encapsulation using a water soluble gum to obtain a semi-permeable water insoluble gel to encapsulate cells for production and other methods of encapsulation are disclosed in U.S. Pat. No. 4,352,883 issued Oct. 5, 1992. U.S. Pat. No. 4,409,331 issued Oct. 11, 1983 discloses a process for production of substances from encapsulated cells, molecular weight cut-off of membranes, use of divalent cations for polymerization, use of various therapeutic substances, core materials, and methods of formation of the gel including cross-linkers.

Shape-retaining gelled masses that expand before membrane formation, and upon contact with chelator can be made to liquefy within the membrane, and having an optional second membrane are disclosed in U.S. Pat. No. 4,663,286 which issued May 5, 1987.

Double-membrane capsules with high molecular weight cut-offs such as 200-400 kD for the inner membrane, enabling higher density growth of encapsulated cells, and use of poly-L-lysine, are disclosed in EPO Publ. No. 0301 777 of Jan. 2, 1989.

U.S. Pat. No. 5,084,350 issued Jan. 28, 1992, discloses gels reliquified within the capsule for a variety of biological samples, and materials for other implantable device components.

Implantable devices with multiple coatings including a halo layer, and not requiring a poly-L-lysine or other polyamino acid or polycation coating are disclosed in WO 95/19743 published Jul. 27, 1995.

Macrocapsular surfaces of decreased surface area and roughness and increased cryoprotectivity, with a variety of co-monomers and free radical initiators of polymerization, are disclosed in U.S. Pat. No. 5,545,423 which issued Aug. 13, 1996.

Methods of the invention can be used with any implantable device that is suitable for delivery and maintenance of biologically active material. Such devices include gel-based implantable devices, for example, the composite implantable devices described herein and in U.S. Pat. No. 5,427,935 (Jun. 27, 1995). However, other devices can be used as well, for example, the devices described in U.S. Pat. No. 4,663,286 (May 5, 1987), particularly, the implantable devices described in U.S. Pat. No. 5,545,423 (Aug. 13, 1996).

Sources of Therapeutic Substances

Implantable devices used in methods of the invention will generally include a source of a therapeutic substance, e.g., a cell or tissue. The source should release a therapeutic substance that is different from the antigens for which the capture agent has specific affinity.

Preferably the source will be one or more living cells. Cells can be growth-inhibited, such that they do not divide, but continue to perform metabolic reactions. Growth inhibition can be achieved by one or more methods known to one with skill in the art, such as irradiation with UV light, by treatment with mitomycin, and by appropriate genetic manipulation. Exemplary cells include pancreatic islets, hepatic cells, neural cells, liver cells, renal cortex cells, vascular endothelial cells, thyroid and parathyroid cells, thymic cells, ovarian cells, blood cells, allografts or xenografts, and genetically engineered cells. Sources of cells and tissues containing cells include, without limitation, tissue or cells removed from a donor animal, tissue or cells of a primary cell culture obtained by incubation or cultivation of donor tissues and cells, cells obtained from viable cell lines and immortalized cell lines, biologically active products of cells or tissues, and the like. Cells from a primary cell line can be treated in culture with one or more cytokines or growth factors. Exemplary cells for transplantation into a subject can be from the same species as that subject, or from a different species that is discordant or concordant with the recipient subject.

In preferred embodiments, the cell is an autologous cell, an allogeneic cell, or a xenogeneic cell. For example, the cell is: an autologous cell, i.e., a cell which is taken from the individual recipient into which the cell will be implanted; an allogeneic cell, i.e., a cell which is taken from a different individual of the same species as the recipient into which the cell will be implanted; a xenogeneic cell, i.e., a cell which is from a different species than the recipient into which the cell will be implanted. In the case of an allogeneic cell, the cell can be fully matched or partially matched for MHC class I loci, fully matched or partially matched for MHC class II loci, and fully matched or partially matched for minor loci. In the case of xenogeneic cells, the cells can be concordant or discordant with respect to the recipient.

In preferred embodiments, the recipient animal is a dog, a pig, or a human. In preferred embodiments the donor cell is a pancreatic islet cell. In preferred embodiments: the composite implantable device contains pancreatic islets, e.g., at e.g., a density of 5,000 to 100,000 islets per milliliter of medium; the composite implantable device contains living cells at a density of about 10⁴ to 10⁸ cells per milliliter of medium.

Implantable devices used in the methods described herein can include a source of a therapeutic substance. For example, the device can include, a composition of matter which produces or releases a therapeutic substance, e.g., a protein, e.g., an enzyme, hormone, antibody, or cytokine, a sense or anti-sense nucleic acid, e.g., DNA or RNA, or other substance which can exert a desired effect on a recipient. The source of a therapeutic substance can be a tissue or a living cell; a eukaryotic cell, e.g., a rodent, canine, porcine, or human cell; a prokaryotic cell, e.g., a bacterial cell; a fungal or plant cell; a cell which is genetically engineered, e.g., a cell which is genetically engineered to produce a protein, e.g., a human protein. The source of a therapeutic substance can be or include an autologous, an allogeneic, or a xenogeneic cell. In the case of an allogeneic cell, the cell can be fully matched or partially matched for MHC class I loci, fully matched or partially matched for MHC class II loci, and fully matched or partially matched for minor loci. In this case of xenogeneic cells, the cells can be concordant or discordant with respect to the recipient.

Implantable devices used in the methods described herein can include a composition of matter which absorbs or modifies or detoxifies a substance produced by the recipient.

Genetically modified cells can be used. This includes cells that have been modified by genetic engineering to produce a product, e.g., cells modified to overproduce a product they normally produce, as well as cells engineered to produce a produce they do not normally make. Cells which have been modified in other ways, e.g., cells modified to reduce an immune response in a subject e.g., cells modified so as not to produce an antigen other than the therapeutic substance, can also be used in methods of the invention.

Isolation of Cells

Living cells can be isolated away from surrounding tissues or grown in culture by procedures known to the art, and then suspended in a liquid medium prior to encapsulation. The living cells can provide biological substances, e.g., enzymes or co-factors, hormones, clotting factors, or growth factors. Cells, e.g., pancreatic cells, can provide enzymatic or hormonal functions. Cells such as hepatic cells can provide a detoxification function.

As an example, pancreatic islet cells were prepared from either adult mongrel dogs, pigs, or bovine calves (0-2 weeks old) by a modification of the methods of Warnock and Rajotte, Diabetes, 37:467 (1988), as previously described in Lanza et al., Proc. Natl. Acad. Sci., 88:11100-11104 (1991).

Briefly, aseptic, viable porcine pancreata were obtained under aseptic operating room procedures. After resection (warm ischemia for less than about 15 minutes), the glands were cannulated and infused with cold (4° C.) University of Wisconsin (UW) organ preservation solution. Pancreatic tissues were dissociated using an intraductal collagenase digestion procedure. The collagenase is delivered by peristaltic pump, and the digested pancreas is mechanically disrupted in a polypropylene dissociation chamber containing 2-6 mm glass beads. The islets were separated from the exocrine tissue by discontinuous density gradient centrifugation (27%, 20.5%, and 11% (w/v) FICOLL® (Sigma, F 9378) in Eurocollins solution).

Isolated islets were then cultured for one day either in M199/Earle's medium supplemented with 10% (vol/vol) fetal bovine serum, 30 mM HEPES, 100 milligrams/dl glucose, and 400 IU/ml penicillin (canine), or in α-MEM plus 10% heat-inactivated horse serum (bovine and porcine) in a humidified atmosphere of 5% CO₂/95% air at 37° C. A typical yield of islets should be in the range of 0.05×10⁶, to 0.1×10, to 0.5×10⁶, to 1.0×10⁶, to 1.8×10⁶ islets for adult pancreas (400 gm wet weight, islet diameter 80-125 μm, purity 85-95%, viability greater than 90%; see below). The cells can also be isolated by other procedures and cultured under other suitable conditions.

Ischemic deterioration of the islet cells is minimized by using tissue fragments of a suitable size, e.g., islet fragments should be less than about 150 microns, and preferably 50 to 125 microns, in diameter. Viability, growth, longevity, and/or function of the islet cells can be enhanced by co-culturing, i.e., by mixing other cell types in the liquid medium prior to encapsulation. Useful cell types include cells that secrete growth hormone, e.g., GH-3 cells, or cells that secrete connective tissue and/or extracellular matrix components, e.g., fibroblasts and endothelial cells. In addition, cells, e.g., islets, can be co-cultured with red blood cells, or hemoglobin, or other oxygen carrying agents can be added, to enhance oxygen availability. Red blood cells can also be used to rescue tissue from damaging effects of nitric oxide.

Islet quality control procedures are used to enable comparison of different lots of islets prepared at different times. Purity (amount of islet tissue compared to exocrine tissue contamination) can be determined by ability of pancreatic islets to rapidly take up diphenyl thiocarbazone (dithizone). Islets can be incubated for five to ten minutes with 50 micrograms/ml of dithizone (D5130, Sigma) to stain them red. The preparation is then examined under light microscopy for a qualitative estimate of purity. Quantification of purity is effected by islet dispersion and counting of stained and unstained cells, or with a spectrophotometric assay of dithizone uptake/micrograms DNA.

Viability can be determined by any one of several assays that depend on the capability of viable cells to exclude certain dyes. For example, one assay uses a combination of the fluorescent stains acridine orange, which stains only viable cells green, and propidium iodide, which stains only the nuclei of dead cells red. The islets are incubated with the dyes (acridine orange, Sigma A6014, 50 micrograms/ml, and propidium iodide, Sigma P4170, 2.5 micrograms/ml) in a PBS solution for 10 to 15 minutes and then dispersed into single cells. Counts of red and green fluorescing cells are used to calculate percent viability.

Insulin secretory activity of the islets is determined both in static culture, e.g., expressed as units of insulin per islet volume, and based on the capability of the islets to respond to graded concentrations of glucose. These values are quantitatively established by measuring the insulin secreted by islets exposed to a range of glucose concentrations extending from 2.8 to 28 milliM glucose.

Formation of Implantable Devices

Living cells, e.g., islet cells, can be encapsulated in a variety of gels, e.g., alginate, to form implantable devices, e.g., microparticles, e.g., microbeads or microspheres to physically isolate the cells once implanted into a host. To prevent entry of smaller molecular weight substances such as antibodies and complement (with a molecular weight of about 150 kDa) into these microparticles, they can be coated with a monodisperse polyamino acid which provides an outer shell with a controlled pore size, or they can be treated by, e.g., cross-linking, to control their internal porosity. Alternatively, their porosity can be controlled by mixing various substances such as polyethylene oxide (PEO) directly into the gel mixture. The use of a high molecular weight molecule, e.g., a high molecular weight PEO, e.g., of about 1-8 million Da, will minimize the escape of the porosity controlling substance. Molecules of this size range can be used with or without an outer coating.

Encapsulation

The description below is directed primarily to the formation of microcapsules, but the cells or other elements, can also be incorporated into other implantable devices, e.g., implantable macroencapsulation-devices, perfusion based devices such as “hockey puck” type devices and extra corporeal devices.

Once the cells are isolated and suspended in liquid medium, they can be encapsulated by a supporting matrix, e.g., a hydrogel matrix to form a microbead, which serves as a core of an implantable device, e.g., or internal particle. The core maintains a proper cell distribution, provides strength, and enhances cell viability, longevity, and function. The core can also contribute to immunoisolation. For example, the physical distance that is created by embedding the internal particle in a supporting matrix, can provide protection from, e.g., nitric oxide and cytokines. It also protects the internal particle from direct cell-cell interactions that can elicit an undesirable host response.

Using standard techniques, a gel matrix is formed by adding cells, e.g., pancreatic islets, to a solution of nutrient medium and liquefied gel, e.g., sodium alginate, to form a suspension, and then crosslinking the gel. A capture agent can be added at this stage. The gel matrix can be any one or a combination of a variety of substances, preferably substances that are biocompatible with the host animal, and are capable of maintaining cellular viability and physically supporting the tissue or cells in suspension.

The gels can be gelled or crosslinked, e.g., by the addition of ions such as calcium, potassium, or barium, or by a change in temperature. If temperature change is used, however, care should be taken to choose appropriate temperature changes for gelation that are not harmful or fatal to the living cells to be encapsulated. Temperature-independent gels include alginates, carrageenans, and gums such as xanthan gum. As used herein, the term alginate includes alginate derivatives. These gels should be treated using standard techniques, to remove polyphenols, lipopolysaccharides, endotoxins, and other impurities.

Alginate is composed of blocks of 1,4 linked β-D-mannuronic acid (M) and α-1-guluronic acid (G) linked together, e.g., in alternating MG blocks. The preferred alginate is one formulated with a high G block content, e.g., at least about 60 percent. The higher the percentage of G blocks in the alginate composition, the greater the pore size and the strength of the gel matrix that is obtained in the final product. In addition, alginate gels with a high M block content appear to be more immunogenic than gels with a high G block content. See, e.g., Soon-Shiong et al., Transplant. Proc., 23:758-759 (1991), and Soon-Shiong et al., Transplantation, 54:769-774 (1992).

The gel matrix should be sufficiently viscous to maintain the cells in a dispersed state. When alginate is used as the gel matrix, it is added up to about 3%, preferably to about 1 to 2%, of the liquid medium, and the solution is cross-linked to form a semisolid gel in which the cells are suspended. These percentages provide a matrix that maintains its shape and has sufficient mechanical strength to remain intact in vivo for several months.

Alginate hydrogels are preferred for the microbead cores for a number of reasons. Alginate allows rapid polymerization and immobilization of cells at room temperature using relatively benign CaCl₂, provides consistent gel rheology that can be conveniently varied by increasing alginate concentration, and produces microbeads with good mechanical strength.

A preferred method for making hydrogel microbeads is with an air jet.

Other methods for making hydrogel microbeads including emulsification, use of an electrosprayer, dripping, and the Rayleigh jet.

Controlling Pore Size of Microparticles

The pore size of the microparticles can be controlled either by applying a selectively permeable shell of a monodisperse polyamino acid having a particular molecular weight cutoff. This can be effected by applying an “electrostatic” coating, e.g., a coating of a polyamino acid, e.g., polylysine. Pore size can also be controlled by treating the gel matrix of the microparticles themselves to change the pore size of the matrix without any subsequent coating. E.g., the surface of the core can be altered by, e.g., cross-linking, to produce covalently modified gel matrix surface. A coating can be a formed by modifying the structure of the matrix, e.g., the matrix can be cross-linked, e.g., with metal ions, e.g., Ba or Fe ions, or by photo-cross-linking, to form a coating.

As used herein, “molecular weight cutoff” refers to the size of the largest molecule that is not substantially blocked, e.g., by a selectively permeable shell or coating surrounding a microsphere or by the gel matrix itself or both. Molecules with a molecular weight above the cutoff are substantially prevented from entering or leaving the particle. The composite implantable device should generally provide a molecular weight cutoff of about 50 kDa, more preferably about 100 kDa, more preferably about 150 kDa, and most preferably about 400,000 daltons. In preferred embodiments, the molecular weight cutoff is sufficient to prevent Ig molecules, e.g., IgG, and complement, from entering and coming into contact with the encapsulated material.

Altering the Pore Size of the Gel Matrix

The pore size of the gel matrix can be altered in several ways. For example, the gel matrix can be altered, e.g., the porosity can be either increased or decreased so as to influence the transport properties, e.g., permeability and/or molecular weight cutoff, by adding, e.g., gelatin, or collagen, or barium, or other ions with the same valance as Ca⁺⁺ ions. Changes in the temperature will also affect the pore size. An increase in the temperature will result in shrinkage of the gel matrix. The addition of compound, e.g., PEO or PEG, to the gel matrix can also result in altered pore size. PEO or PEG can act to repel protein and to hinder fibrotic response. In preferred embodiments, PEO of molecular weight greater than 1,000 kDa, more preferably greater than 4,000 kDa, and most preferably greater than 8,000 kDa, is mixed with the gel matrix. PEO of relatively high molecular weight will not diffuse out and thus does not require crosslinking.

Coatings that Minimize Particle Volume

Embodiments of the invention use monodisperse coatings that reduce the volume of a component, e.g., a core, to which they are applied. For example, a monodisperse polyamino acid coating, e.g., a polylysine, or polyornithine made from a polyamino acid of a relatively low molecular weight, can result in a significant reduction in the volume of a gel core, e.g., an alginate core, to which it is applied. In many cases the reduction in volume is as much as about 50%, or even 60-70%, or more.

Relatively low molecular weight, as used herein, means about 30,000 Da or less, more preferably about 15 kDa or less, more preferably about 10 kDa or less, more preferably about 8 kDa or less, more preferably about 7 kDa or less, more preferably about 5 kDa or less, more preferably about 4 kDa or less, more preferably about 3 kDa or less, and most preferably about 1.5 kDa or less.

For example, the use of monodisperse polylysine of a relatively low molecular weight, e.g., 3, 7, or 9.6 kDa, can result in a significant reduction, (approximately 30% in some cases) in the diameter, of the core to which it is applied. In addition to the decrease in volume, the use of a low molecular weight polyamino acid will result in a coat having superior permselective properties. However, the use of a low molecular weight polyamino acid often results in a surface which is “pruned”, i.e., relatively convoluted or rough, and which can elicit a fibrotic response. The composite implantable device of the invention, by using this coating on the internal particle, and a smooth surface, e.g., of alginate, on the exterior of the composite implantable device, can obtain the benefits of a coating of relatively low molecular weight and also inhibit fibrosis.

The permselectivity properties of a polyamino acid, e.g., a polylysine, coating improve after the coating has been aged 2 or more hours. Thus, for best results, particles coated with these coatings should not be implanted in recipients until the coating has aged.

Geometric Stabilization

Some particles or components are not geometrically stable immediately after manufacture, e.g., the particle or component can change size or shape. If internal particles which are incorporated into a composite implantable device change geometry, the components of the composite implantable device, e.g., the super matrix or outer coating, can be damaged and the integrity of the composite implantable device can be compromised. Although not wishing to be bound by theory, the inventors believe that changes in the geometry can damage the super matrix or the outer coating, e.g., by inducing fissures or discontinuities. Damaged particles can allow the fibrotic proliferation of host cells on the inner particles when implanted into a host. Therefore, it is often desirable to geometrically stabilize internal particles, preferably prior to incorporating them into composite implantable devices. Stabilization can generally be accomplished by allowing the particles to “age” for a short time before incorporation into larger structures. The aging should be done under condition which maximizes the viability of encapsulated cells. Geometric stabilization is particularly important when the particles are coated with a relatively low molecular weight poly-amino acid.

Polylysine-coated alginate particles, especially those coated with relatively low molecular weight polylysine, should be geometrically stabilized. The polylysine coated alginate particles should be placed in a culture medium, suitable for the cell being used, and allowed to stabilize overnight. Geometric stabilization methods taught in PCT/US96/03135 and U.S. patent application Ser. No. 08/402,209, Filed Mar. 10, 1995, can be used with methods and devices of the invention.

Improved Three-Part Composite Selectively Permeable Coatings

The inventors have discovered a particularly efficacious selectively permeable coating to be used in devices of the invention, in particular implantable devices, and in particular microreactors.

Accordingly, in another aspect, the invention features, a selectively permeable coating which includes a first, or innermost layer of a monodisperse polyamino acid; an intermediate gel component, which can be a thin discrete layer but more preferably is integrated into one or both of the polyamino acid layers; and a second or outermost layer of a monodisperse polyamino acid. This coating is referred to herein as a three-part composite layer. Preferably the layers are adjacent to one another in contact and are not spaced apart. In preferred embodiments the polyamino acid layers are within 10, 5 or 1 or 2 μm of each other and are more preferably in contact with one another.

The molecular weight of the inner layer of monodisperse polyamino acid should be chosen such that it is lower than the molecular weight of the outer layer of monodisperse polyamino acid. Preferably, the inner most layer is in the range of 1-5 kDa, more preferably 2-4 kDa. In preferred embodiments the molecular weight is about 2 kDa or 3.9 kDa. (The molecular weight can be determined by as average molecular weight by viscosity or by Lall's method). Generally the inner layer is chosen to optimize permselectivity, e.g., the ability to exclude IgG. Generally, lowering the molecular weight improves selectivity. However, the use of relatively low molecular weights also has disadvantages, e.g., the use of low molecular weight coatings can result in mechanical instability and the induction of faults or fissures in other components of the microreactor, e.g., in the matrix or super matrix of a microreactor. Such fissures or faults can allow the development of fibrotic proliferation on the surface of an internal particle or a particle which has induced faults in the surrounding matrix or super matrix. The disadvantages accompanying the use of such low molecular weight coatings can be overcome by adding additional members or components to the coating, an intermediate alginate member and second, outermost coating of a polyamino acid. The molecular weight of the inner layer of polyamino acid should sufficiently small that without the other two members of the three part layer it would induce faults in a surrounding matrix.

The intermediate member, is a gel, e.g., alginate. It is an electrostatic component which often does not form a discrete layer between the two monodisperse polyamino acid layers but rather integrates into the two. If a discreet layer is formed it is thin, e.g., it is no more than 10, more preferably no more than 5, or 1 or 2 μm in thickness. The gel should be such that it can electrostatically saturate the underlying polyamino acid layer and does not exceed the thickness limitations described herein.

The outer layer of monodisperse polyamino acid has a higher molecular weight than the inner layer. While molecular weight is minimized in the first layer to maximize permselectivity (i.e., selective permeability) a higher molecular weight is used in the outer layer to promote mechanical stability and to prevent the induction of fissures or faults in the surrounding gel matrix or supermatrix. In preferred embodiments the molecular weight is between 5 and 15 and more preferably between 5-15, 8-12, and 9-10 kDa. In particularly preferred embodiments the molecular weight is about 9 kDa. The molecular weight of the outer layer of polyamino acid should be sufficiently large that in combination with the other two elements, faults are not induced in a surrounding matrix.

In preferred embodiments, the coating includes an inner layer of size-homogenous polyamino acid, preferably polylysine, of about 2-4 kDa; an intermediate gel member, preferably alginate, which integrates into the inner polyamino acid layer; an outer layer of a monodisperse polyamino acid, preferably, polylysine, of about 8-12 kDa.

This three-part composite layer can be used on any of the devices described herein. In preferred embodiments the three-part composite layer coats: an internal particle of a single composite microreactor; an internal particle of a double composite microreactor; a particle of a double composite microreactor; an internal particle and a particle of a double composite microreactor.

Composite Microreactors

Structural Components

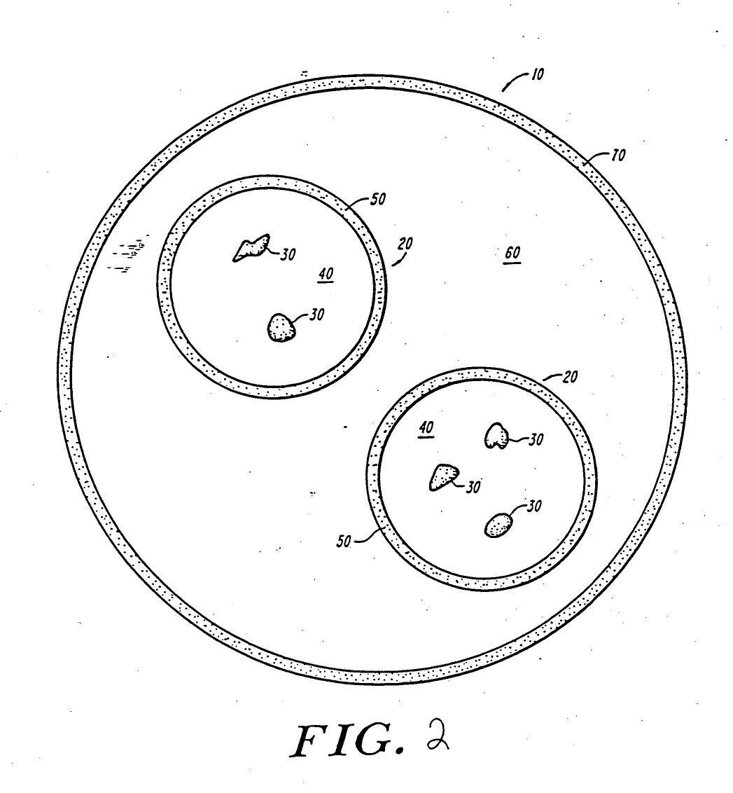

FIG. 2 is a schematic diagram of a simple or single composite microreactor (10). The composite microreactor (10) contains at least one, and preferably a plurality of internal particles (20). An internal particle (20) includes one or a plurality of sources (30) of a therapeutic or otherwise desirable substance. The sources (30) are embedded carried on, adhered to, or in an internal particle matrix (40). The internal particle (20) can optionally include an internal particle coating (50). The internal particles can be embedded in a super matrix (60). The composite microreactor 10 can (optionally) include an outer coating (70).

The diameter of the composite microreactor (10) is preferably between 100 microns and 4 millimeters, between 300 and 1200 microns, between 300 and 1500 microns, between 400 and 1000 microns, or between 400 and 800 microns. More preferably the diameter is about 600 microns.

The diameter of the internal particles (20) before application of a volume-reducing coating (described below) is preferably between 50 and 700 microns, more preferably between 100 and 500 microns, more preferably between 200 and 400 microns, and most preferably about 300 microns in diameter. The diameter of the internal particles (20) after application of a volume-reducing coating (described below) is preferably between 35 and 500 microns, more preferably between 75 and 400 microns, more preferably between 100 and 300 microns, and most preferably about 200 microns in diameter.

As is discussed in more detail elsewhere herein, the source 30 can be a cell, or a group of cells, e.g., an islet. The sources of an internal particle can all be of one type or more than one type of source can be included in an internal particle. Furthermore, the composite microreactor 10 can include more than one type of internal particle (20), e.g., the composite microreactor 10 can include a first type of internal particle (20) having within it a first source, e.g., a first type of cell, and a second type of internal particle (20) having within it a second source, e.g., a second type of cell.

The internal particle matrix (40) can be a gel, e.g., a hydrogel, e.g., alginate or agarose. The internal particle matrix can also be a solid particle, e.g., a glass bead, or a porous structure, on which anchorage dependent cells can be seeded. The internal matrix (40) can have immunoisolative properties. In some embodiments it has little or no ability to exclude low molecular weight species, e.g., release of source antigens or invasion by IgG or complement, with this property being relegated to other components of the composite microreactor (10). The internal particle matrix (40) can be rendered immunoisolating by controlling its porosity, e.g., such that it hinders the passage of molecules of relatively large molecular weight, or by adding to it components, e.g., polyethylene oxide (PEO), polystyrene sulfonic acid (PSA), polyethylene glycol (PEG), or polyornithine (PLO) which hinder the passage of molecules of relatively large molecular weight. Regardless of the method of controlling its permeability, the internal matrix (40), in preferred embodiments will hinder the passage, and preferably, essentially completely prevent the passage of: molecules having a molecular weight of more than about 400 kDa, preferably more than about 150 kDa, preferably more than about 100 kDa, and most preferably more than about 50 kDa; or of immune system components such as Ig molecules or complement; or recipient-derived cells. The internal matrix (40) need not be anti-fibrotic and need not be biocompatible. The composite microreactor (10) can include more than one type of internal particle (20), e.g., the composite microreactor (10) can include a first type of internal particle (20) having within it a first type of internal matrix (40) and a second type of internal particle (20) having within it a second type of internal matrix (40).

The internal particle coating (50) is optional. It can be made of a monodisperse polyamino acid, e.g., polylysine (PLL), or polyorinthine (PLO). In addition, the coating can be a formed by modifying the structure of the matrix, e.g., the matrix can be cross-linked, e.g., with metal ions, e.g., Ba or Fe ions, or by photo-cross-linking, to form a coating. A preferred coating is monodisperse polylysine having a molecular weight of less than 15 kDa, more preferably of less than 10 kDa, more preferably of less than 5 kDa. Particularly preferred are monodisperse polylysines with a molecular weight of about 3-4 kDa, e.g., 3.7 kDa, or about 9-10 kDa, e.g., 9.7 kDa. Preferred coatings are volume-reducing coatings. Furthermore, the composite microreactor (10) can include more than one type of internal particle (20), e.g., the composite microreactor (10) can include a first type of internal particle (20) having a first type of internal particle coating and a second type of internal particle (20) having a second type of internal particle coating (50). Because, in some embodiments, the internal particles coating (50) need not be biocompatible and need not be anti-fibrotic, other properties of the internal coating (50), e.g., its ability to immunoisolate, can be optimized without the necessity of any compromise to confer biocompatibility or anti-fibrotic activity.

Super matrix (60) can be a gel, e.g., a hydrogel, e.g., alginate or agarose. The super matrix (60) can have immunoisolative properties. In some embodiments it can have little or no ability to exclude low molecular weight species, e.g., IgG or complement, with this property being relegated to other components of the composite microreactor (10). The super matrix (60) can be rendered immunoisolating by controlling its porosity, e.g., such that it hinders the passage of molecules of relatively large molecular weight or by adding to it components, e.g., PEO, PSA, PEG, or PLO which hinder the passage of relatively large molecules. Regardless of the method of controlling its permeability, the super matrix (60) will in preferred embodiments, will hinder the passage, and preferably essentially completely prevent the passage of: molecules having a molecular weight of more than about 50 kDa, preferably more than about 100 kDa, preferably more than about 150 kDa, and most preferably more than about 400 kDa; or of immune system components such as Ig molecules or complement; or recipient-derived cells. The super matrix (60) need not be anti-fibrotic and need not be biocompatible if a more proximal or more exterior component supplies these functions.

A capture agent can be included on the surface of an internal particle (50). A preferred location for a capture agent is the super matrix (60) and an even more preferred location is the outer coating (70). Positioning on the super matrix (60) or on the outer surface (70) allows binding of source antigen molecules before they are released from the microreactor.

Outer coating (70) is optional. It can be made of a monodisperse polyamino acid, e.g., PLL or PLO. Alternatively, the coating can be a formed by modifying the structure of the matrix, e.g., the matrix can be cross-linked, e.g., with metal ions, e.g., Ba or Fe ions, or by photo-cross-linking, to form a coating. A preferred coating is monodisperse polylysine having a molecular weight of less than 15 kDa, more preferably of less than 10 kDa, more preferably of less than 5 kDa. Particularly preferred are monodisperse polylysines with a molecular weight of about 3-4 kDa, e.g., 3.7 kDa, or about 9-10 kDa, e.g., 9.7 kDa. Preferred coatings are volume-reducing coatings. The outer coating (70) need not be immunoisolating if other components supply this function.

The multi-component structure of the composite microreactor allows selection of materials that can optimize performance. Coating materials that are highly immunoisolating, but less desirable in terms of their biocompatibility or anti-fibrotic activities, can be used in the internal particle coating. The multi-component structure also allows for multiple lines of defense against invasion by recipient immune system components. E.g., the use of an outer coating which releases 1 in 10² source antigen molecules, a super matrix which releases 1 in 10² source antigen IgG molecules, and an internal particle coating which releases 1 in 10² source antigen molecules, results in a composite release rate of only about 1 in 10⁶ of the source antigen molecules.

The multi-component structure of composite reactors also allow a rescue agent to be placed in a zone or compartment between the source of a therapeutic substance and the host.

The ability to segregate functions also allows construction of composite microreactors the life of which are roughly commensurate with the useful life of the enclosed sources. For example, gelatin, which weakens the matrix, could be added. If it is necessary to strengthen the matrix, fibers can be added.

A preferred composite microreactor is one in which: the composite microreactor contains at least two internal particles; the source of a therapeutic or otherwise desirable substance is a cell, e.g., an islet cell; the internal particle matrix is alginate; the internal particle includes an internal particle coating of monodisperse polylysine; the internal particles are embedded in a super matrix of alginate; and the composite microreactor includes an outer coating of monodisperse polylysine; the monodisperse polylysine of the internal particle coating is of a molecular weight of between 2 and 10 kDa; the monodisperse polylysine of the outer coating is of a molecular weight of between 2 and 10 kDa; the internal particles are geometrically stabilized, as is described below; the composite microreactor is generally stabilized, as is discussed in PCT/US96/03135 and U.S. patent application Ser. No. 08/402,209, filed Mar. 10, 1995; the super matrix is free of fissures or other defects which arise the use of internal particles which have not been geometrically stabilized; the diameter of an internal particle, is between 100 and 400 microns, preferably about 200 microns; the diameter of the composite microreactor is between 400 and 800 microns, preferably about 600 microns, and a capture agent is incorporated into the super matrix.

As described above, the internal particle (20) can consist of sources embedded in a matrix, the matrix being enclosed in an internal particle coating. The internal particle (20) can also have other structures. For example, the inner particle can consist of a solid bead, e.g., a plastic bead, a Sepharose bead, (Pharmacia, Piscataway, N.J.) or a glass bead, on which cells, e.g., anchorage dependent cells, are allowed to grow. Cells can be allowed to grow on a surface of the solid bead or, if they are present, within interstitial spaces of the bead. Such an internal particle can be coated as described herein, or left uncoated. The internal particle can be coated or left uncoated. The internal particle, can be embedded in a super matrix, the super matrix being enclosed by coating.

Composite microreactors can also contain fibers or materials to enhance the mechanical strength of the sphere. Similarly, the composite material can contain substances such as PEO or PEG which can act to repel protein and to hinder the fibrotic response. Other materials such as gelatin or collagen can also be added to either increase or decrease the porosity so as to influence the transport properties (permeability/and or molecular weight cutoff).

In addition to advantages, such as ease of retrieval, the embodiments of the invention permit the use of immunoprotectants which are not biocompatible. Materials which alone might be digested by enzymes, or which might trigger a fibrotic response when they come into direct contact with host tissues can be used to form permselective barriers. Methods of the inventor can also be used to alginate-coat particles made of neutral or positively-charged substances. More importantly, the alginate coating furnishes the composite structure with a physical barrier of substantial thickness versus the “coating” formed by mere electrostatic interactions. The composite structure (ranging in diameter from <50 μm to >5 mm) can be made of any material.

Internal particles can be of any shape, including, for example, planar, cubical, tubular, and disk-shaped particles and chambers, or other shapes which might otherwise become fibroencapsulated.

The ratios of the volume of the internal particles to the volume of the composite microreactor can be tailored to particular applications, but preferred ratios are 0.5:5.0, preferably 1.0:3.5, more preferably 1.0:2.5, or 1.0:3.0.

Higher Order Composites

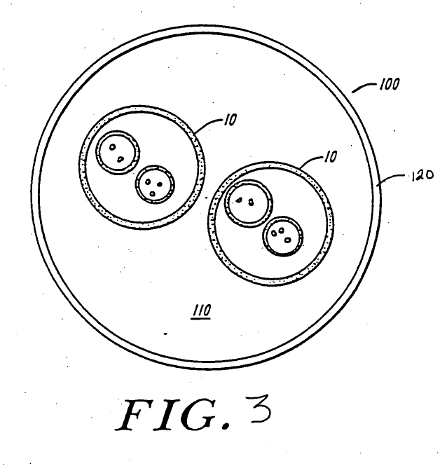

Embodiments of the invention include higher order composite microreactors, e.g., a double composite in which single composite microreactors (10) are embedded in a matrix which is (optionally) coated with an outer coating. Accordingly, FIG. 3, shows a second order, or double composite microreactor (100).

The double composite microreactor (100) contains one or a plurality of composite microreactors (10) (as described above and elsewhere herein) embedded in a double composite microreactor matrix or super matrix (110) which is (optionally) enclosed in a double composite microreactor outer coating (120).

The diameter of the double composite microreactor (100) is preferably between 100 microns and 4 millimeters, between 300 and 1500 microns, between 400 and 1000, or between 500 and 900 microns. More preferably the diameter is about 600-800 microns.

Double composite microreactor matrix or super matrix (110) can be a gel, e.g., a hydrogel, e.g., alginate or agarose. The double composite microreactor matrix or super matrix (110) can have immunoisolative properties. In some embodiments it can have little or no ability to exclude low molecular weight species, e.g., IgG or complement, with this property being relegated to other components of the composite microreactor (10). The double composite microreactor matrix or super matrix (110) can be rendered immunoisolating by controlling its porosity, e.g., such that it hinders the passage of molecules of relatively large molecular weight or by adding to it components, e.g., PEO, PSA, PEG, or PLO which hinder the passage of relatively large molecules. Regardless of the method of controlling its permeability, the matrix or super matrix (110) will, in preferred embodiments will hinder the passage, and preferably, essentially completely prevent the passage of molecules having a molecular weight of more than about 400 kDa, preferably more than about 150 kDa, preferably more than about 100 kDa, and most preferably more than about 50 kDa; or of immune system components such as Ig molecules or complement; or recipient-derived cells. The double composite microreactor matrix (110) need not be anti-fibrotic and need not be biocompatible if a more proximal or more exterior component supplies these functions. In double composite, the matrix of the inner most particle is usually referred to as the internal particle matrix. The matrix in which the internal particles are embedded is usually referred to as the particle matrix, and the matrix in which the single composite particles are embedded is usually referred to as the super matrix. A capture molecule can be covalently coupled to a super matrix molecule.

Double composite microreactor outer coating (120) is optional. It can be made of a monodisperse polyamino acid, e.g., PLL or PLO. A preferred coating is a monodisperse polyamino acid, e.g., polylysine, having a molecular weight of less than 15 kDa, more preferably of less than 10 kDa, more preferably of less than 5 kDa. Particularly preferred are monodisperse polylysines with a molecular weight of about 3-4 kDa, e.g., 3.7 kDa, or about 9-10 kDa, e.g., 9.7 kDa. Preferred coatings are volume-reducing coatings. The double composite microreactor outer coating (120) need not be immunoisolating if other components supply this function. A capture agent can be covalently coupled to an outer coating or component.

Other embodiments of the invention include higher order composite microreactors, e.g., third order composites which include double composite microreactors embedded in a matrix and (optionally) enclosed in an outer coating, and forth order composites, which includes third order composites embedded in a matrix and (optionally) enclosed in an outer coating. The materials and methods discussed for use in simple and double composites can be used for the matrices and coatings of higher order composites.

The multicomponent structure of double (or higher order) composites also allows a capture agent to be placed in a zone or compartment which is interposed between the source of a therapeutic substance and the host. E.g., a source of a therapeutic substance can be disposed within the internal particle and one or more capture agents disposed in one or more of the particle matrix or the super matrix zones, or on the outer coating.

Formation of Composite Microreactors

Composite microreactors can be made by materials which are analogous with the methods used to make internal particles: individual or small numbers of internal particles (rather than cells) are embedded in a matrix (referred to herein as a super matrix to distinguish it from the internal particles matrix) and an (optional) outer coating applied.

For example, after the internal particles are prepared and, e.g., either coated or otherwise treated, e.g., cross-linked, they should be washed in medium to prevent the existing microparticles from sticking to each other (particles that have not been coated should be washed in calcium and magnesium free medium), mixed with a liquid hydrogel such as alginate, and formed into a composite microparticle with a diameter from less than 50 μm up to more than 5 mm. For example, in a method which is analogous to that described above for the creation of the internal particles, a mixture of internal particles in a liquid gel can be extruded through an 18 gauge catheter to form composite microreactors.

As is discussed elsewhere herein, it may be desirable to geometrically stabilize the internal particles before incorporating them into a composite microreactor.

The super matrix of a composite microreactor can provide a selectively permeable shell of a hydrogel material around all of the encompassed internal particles can provide a physical barrier of substantial thickness compared to the individual coatings on each of the microcapsules. Electrostatic interactions in the super matrix can contribute to immunoisolation.

The super matrix can be made of the same material as the internal particle matrix or it can differ from the matrices of some or all of the internal particle matrices.

A composite microreactor can contain internal particles of any shape, including, for example, planar, cubical, tubular, and disk-shaped particles and chambers, which might otherwise become fibroencapsulated.

A composite microreactor can also contain other substances to modify the properties of the composite microreactor and can, e.g., include fibers or materials in addition to the hydrogel matrix and internal particles to enhance the mechanical strength of the composite microreactor. Similarly, the composite microreactors and particularly the super matrix, can include substances such as PEO or PEG which act to repel proteins and to hinder the fibrotic response. Other materials such as gelatin or collagen can also be added to the super matrix to either increase or decrease the porosity so as to influence the transport properties (permeability and/or molecular weight cutoff) of the composite microreactors.

Higher order composites can be made by analogous methods.

Outer Coating

Composite microreactors can (optionally) be provided with an outer coating of a monodisperse polyamino acid. Because the various properties needed by the implanted device, e.g., biocompatibility, the ability to resist fibrotic encapsulation, the ability to prevent recipient immune inactivation of the implanted donor tissue, can be distributed among the various components of the composite microreactor, the outer coating need not supply all of these properties. It may be desirable to geometrically stabilize the supermatrix prior to application of a coating.

Exemplary Microreactors

In one aspect, the invention features, a composite microreactor which includes:

(a) one, or a plurality of an internal particle which includes:

(i) a source of a therapeutic substance, e.g., an islet;

(ii) an internal particle matrix, e.g., a gel core or a solid particle, which contacts the source;

(iii) (optionally) an internal selectively permeable particle coating of a monodisperse polyamino acid enclosing the internal particle matrix; and

(b) a super matrix, e.g., a gel super matrix, in which the internal particle (or particles) is embedded; and

(c) (optionally) an outer selectively permeable coating of a monodisperse polyamino acid enclosing the super matrix, the composite microreactor preferably providing a molecular weight cutoff that prevents molecules larger than about 400,000 daltons from coming into contact with the source.

In preferred embodiments: an internal particle is coated with the three-part composite layer described herein.

In preferred embodiments, the internal particle matrix is or includes: a gel, e.g., a hydrogel, e.g., an alginate or agarose gel; a solid particle, e.g., a glass bead; a particle having pores or interstices. In preferred embodiments the internal particle matrix is other than a liquid. The internal particle matrix can include substances which impede the passage of recipient-derived molecules or cells, e.g., it can include polyethylene oxide (PEO), or polyethylene glycol (PEG), or polystyrene sulfonic acid (PSA). In preferred embodiments, a capture agent indisposed within the internal particle matrix.

In preferred embodiments, the internal particle matrix hinders the passage, and preferably essentially completely prevents the passage of: molecules having a molecular weight of more than about 400 kDa, preferably more than about 150 kDa, preferably more than about 100 kDa, and most preferably more than about 50 kDa; or of immune system components such as Ig molecules or complement.

In preferred embodiments, the internal particle coating is or includes: a polyamino acid, e.g., monodisperse polylysine (PLL) or monodisperse polyomithine (PLO). A particularly preferred coating is monodisperse polyamino acid. e.g., monodisperse polylysine or monodisperse polyornithine, having a molecular weight of less than 15 kDa, more preferably of less than 10 kDa, more preferably of less than 5 kDa. Particularly preferred are monodisperse polyamino acids, e.g., monodisperse polylysines, with a molecular weight of about 1 kDa-4 kDa, about 1 kDa-less than 4 kDa, e.g., 3.7 kDa, or about 5 kDa to less than about 15 kDa, or about 5 kDa to less than about 10 kDa, e.g., 9 kDa-10 kDa, e.g., 9.7 kDa. Also preferred are monodisperse polyamino acids, e.g., PLL or PLO, coatings in the range of 1 or 2-10 kD, preferably in the range of 1-2, 1-3, or 1-4 kDa. Preferred coatings are volume-reducing coatings.

In preferred embodiments the internal particle coating hinders the passage, and preferably essentially completely prevent the passage of: molecules having a molecular weight of more than about 400 kDa, preferably more than about 150 kDa, preferably more than about 100 kDa, and most preferably more than about 50 kDa; or of immune system components such as Ig molecules or complement; or host-derived cells.

In preferred embodiments, the super matrix is or includes: a gel, e.g., a hydrogel, e.g., an alginate or agarose gel. In preferred embodiments the super matrix is other than a liquid. The super matrix can include substances which impede the passage of recipient-derived molecules or cells, e.g., it can include polyethylene oxide (PEO), polystyrene sulfonic acid (PSA), or polyornithine (PLO). A high molecular weight, molecule, e.g., a polymer, e.g., PEO, with a molecular weight of 1-8 million daltons, or more, can be added to the super matrix to confer immunoisolating properties.

In preferred embodiments, the super matrix hinders the passage, and preferably essentially completely prevent the passage of: molecules having a molecular weight of more than about 400 kDa, preferably more than about 150 kDa, preferably more than about 100 kDa, and most preferably more than about 50 kDa; or of immune system components such as Ig molecules or complement; or recipient-derived cells.

In preferred embodiments, the super matrix has little or no ability to exclude low molecular weight species, e.g., IgG or complement, with this property being relegated to other components of the microcapsule.

In preferred embodiments, the outer coating (which is optional) is or includes: a polyamino acid, e.g., monodisperse polylysine (PLL) or monodisperse polyornithine (PLO). A particularly preferred coating is monodisperse polyamino acid, e.g., monodisperse polylysine or monodisperse polyornithine, having a molecular weight of less than 15 kDa, more preferably of less than 10 kDa, more preferably of less than 5 kDa. Particularly preferred are monodisperse polyamino acids, e.g., monodisperse polylysines, with a molecular weight of about 1 kDa-4 kDa (or about 1 kDa-less than 4 kDa) e.g., 3.7 kDa, or about 5 kDa to less than 15 kDa, or about 5 kDa to less than about 10 kDa, e.g., 9 kDa-10 kDa, e.g., 9.7 kDa. Also preferred are monodisperse polyamino acid, e.g., PLL or PLO, coatings in the range of 1 or 2-10 kDa, preferably in the range of 1-2, 1-3, or 1-4 kDa.

In preferred embodiments, the outer coating hinders the passage, and preferably essentially completely prevent the passage of: molecules having a molecular weight of more than about 400 kDa, preferably more than about 150 kDa, preferably more than about 100 kDa, and most preferably more than about 50 kDa; or of immune system components such as Ig molecules or complement; or host-derived cells.

In preferred embodiments, the diameter of the internal particle, before application of a volume-reducing coating, is between 50 and 700 microns, more preferably between 100 and 500 microns, more preferably between 200 and 400 microns, and most preferably about 300 microns in diameter. The diameter of the internal particles, after application of a volume-reducing coating, is preferably between 35 and 500 microns, more preferably between 75 and 400 microns, more preferably between 100 and 300 microns, and most preferably about 200 microns in diameter.

In preferred embodiments, the diameter of the composite microreactor is between 100 microns and 4 millimeters, between 300 and 1200 microns, between 300 and 1500 microns, between 400 and 1000 microns, or between 400 and 800 microns. More preferably the diameter is about 600 microns.

In preferred embodiments, the composite microreactor includes one or a plurality of internal particles, e.g., between 1 and 100, e.g., 1 and 10, internal particles.

In preferred embodiments, the composite microreactor is a component of a higher order composite, e.g., a double composite, or a third order composite.

In preferred embodiments, one or more components of the composite is geometrically stabilized, as is taught in PCT/US96/03135 and U.S. patent application Ser. No. 08/402,209, Filed Mar. 10, 1995. For example: the internal particle matrix is geometrically stabilized, e.g., by allowing it to age for between 2 hours and 14 days, e.g., for between 12 hours and 4 or 5 days, prior to coating it; the internal particle is geometrically stabilized, e.g., by allowing it to age for between 2 hours and 14 days, e.g., for between 12 hours and 4 or 5 days prior to embedding it in the super matrix.

In preferred embodiments, the internal particle coating has a lower molecular weight exclusion number than does the super matrix, the outer coating (if present), or the combination of the super matrix and the outer coating, e.g., the internal particle coating excludes recipient immune molecules, e.g., IgG or complement, and the super matrix, the outer coating (if present), or the combination of the super matrix and the outer coating, allows immune molecules, e.g., IgG or complement, to pass but excludes the passage of recipient cells.

In preferred embodiments, the outer surface of the composite is biologically compatible, e.g., it is sufficiently smooth that it inhibits fibrotic encapsulation of the composite; the outer surface of the composite is biologically compatible, e.g., it is sufficiently smooth that it inhibit fibrotic encapsulation of the composite but the surface of the internal particle is not biologically compatible, e.g., it is not sufficiently smooth to inhibit fibrotic encapsulation.

In preferred embodiments, at least one of the super matrix and the outer coating prevents contact of fibrotic cells with the internal particle coating.

In preferred embodiments the composite microreactor further includes:

(b) one, or a plurality, of a second internal particle which includes:

(i) a second source of a therapeutic substance, e.g., an islet or a cell other than and islet;

(ii) a second internal particle matrix which includes the second source,

(iii) (optionally) a second internal particle coating of a monodisperse polyamino acid enclosing the second internal particle;

In preferred embodiments, super matrix prevents contact of fibrotic cells with the internal particle coating; the super matrix and the outer coating (if present) is free of defects which arise from the inclusion of non-geometrically stabilized components, e.g., non-geometrically stabilized internal particles; at least two, or three, or four, components chosen from the group of the internal particle matrix, the internal particle coating, the super matrix, and the outer coating (if present), provides a molecular weight cutoff that prevents molecules larger than about 150,000 daltons from coming into contact with the sources; the internal particle molecular weight cutoff is provided by a pore structure of the internal particle matrix, and that pore structure results, e.g., from cross-linking of the internal particle gel; the molecular weight cutoff the super matrix is provided by a pore structure of the super matrix.

Preferred embodiments lack an outer coating.

In preferred embodiments, the outer surface of the composite microreactor is a gel, e.g., an alginate gel. In more preferred embodiments the outer surface of the composite microreactor is a gel, e.g., an alginate gel, the outer surface of which has been modified, e.g., by cross-linking, to produce a covalently modified gel surface, e.g., to form a coating.

In preferred embodiments, the outer component of the composite microreactor, i.e., the component in contact with the recipient, is at least 50, 75, 90, 95, 97, or 98%, water.

In preferred embodiments, one or more components of the composite microreactor is of sufficient diameter, or of sufficient thickness, such that it imposes a substantial distance (or separation) between recipient cells, e.g., lymphocytes, macrophages, or NK cells, and the source of a therapeutic substance. In more preferred embodiments the thickness (e.g., the distance between its inner surface and its outer surface) of a component, e.g., a matrix, e.g., a particle matrix or super matrix, is: at least 5, 10, 20, 50, 75, 100, or 200 microns. In more preferred embodiments the distance between recipient cells and the source of a therapeutic substance is: at least 5, 10, 20, 50, 75, 100, or 200 microns; sufficient such that exposure of the source of a therapeutic substance to small molecules (e.g., molecules which are not excluded by a component which excludes IgG, e.g., cytokines, nitric oxide (NO), and other toxic moieties) released by recipient cells is substantially reduced (e.g., by diffusion), e.g., reduced at least 10, 20, 50, 75, or 90%; sufficient such that the concentration of small molecules (e.g., molecules which are not excluded by the selectively permeable barrier-components of the composite microreactor, e.g., cytokines, NO, and other toxic moieties) released by recipient cells is substantially reduced (e.g., by diffusion), e.g., reduced at least 10, 20, 50, 75; or 90% at the source of a therapeutic substance. In more preferred embodiments: the distance is supplied by one or both of the particle matrix and the super matrix.

In preferred embodiments, one or more components of the composite microreactor is of sufficient diameter, or of sufficient thickness, such that it imposes a substantial distance (or separation) between recipient cells, e.g., lymphocytes, macrophages, or NK cells, and one or both of the source of a therapeutic substance or donor antigen (other than the therapeutic substance) released by the source (e.g., donor proteins, which could stimulate a recipient response against donor tissue). In more preferred embodiments the thickness (e.g., the distance between its inner surface and its outer surface) of a component, e.g., a matrix, e.g., a particle matrix or super matrix, is: at least 5, 10, 20, 50, 75, 100, or 200 microns. In more preferred embodiments the distance between recipient cells and the source of a therapeutic substance is: at least 5, 10, 20, 50, 75, 100, or 200 microns; sufficient such that the amount, number, or concentration of a donor antigen, released into the recipient, or contacting recipient cells, is substantially reduced (e.g., by diffusion, or by trapping in or exclusion by the component or components which supply the separation), e.g., reduced by at least 10, 20, 50, 75, or 90%; sufficient such that contact of cells of the recipient with donor antigens, e.g., proteins, which protrude from or extend through the internal particle matrix or internal particle coating, or both, is substantially reduced (e.g., by diffusion, or by trapping in or exclusion by the component or components which supply the separation), e.g., reduced by at least 10, 20, 50, 75, or 90%; sufficient to inhibit acute release of donor antigens. In more preferred embodiments: the distance or separation is supplied by one or both of the particle matrix and the super matrix.

The inventors have discovered that a single or simple composite microreactor, e.g., one which includes one or more microcapsules contained in a larger particle, can be used to immunoisolate donor tissue. They have further discovered that higher order composites, e.g., double composites, which include one or more single composites contained in a larger particle, and which include a capture agent, are also effective for immunoisolating donor tissue.

Accordingly, the invention features, a double composite microreactor which includes:

(1) one, or a plurality, of an internal particle which includes:

(a) a source of a therapeutic substance, e.g., an islet;

(b) an internal particle matrix which contacts the source; and

(c) (optionally) an internal particle selectively permeable coating of a monodisperse polyamino acid enclosing the first internal particle matrix;

(2) one, or a plurality, of a particle which includes:

(a) the internal particle or particles of (1)

(b) a particle matrix in which the internal particle (or internal particles) is embedded; and

(c) (optionally) a particle selectively permeable coating of a monodisperse polyamino acid enclosing the particle;

(3) a super matrix in which the particle (or particles) of (2) is embedded; and

(4) (optionally) a super matrix or outer selectively permeable coating, e.g., of a monodisperse polyamino acid, e.g., polylysine, enclosing the super matrix.

In a preferred embodiment, the internal particle matrix is or includes: a gel, e.g., a hydrogel, e.g., an alginate or agarose gel; a solid particle, e.g., a glass bead; a particle having pores or interstices. In preferred embodiments the internal particle matrix is other than a liquid. The internal particle matrix can include substances which impede the passage of recipient-derived molecules or cells, e.g., it can include polyethylene oxide (PEO), polystyrene sulfonic acid (PSA), polyethylene glycol (PEG) or polyomithine (PLO).

In preferred embodiments: an internal particle is coated with the three-part composite layer described herein; a particle is coated with the three-part composite layer described herein; an internal particle and a particle are coated with the three-part composite layer described herein; an internal particle does not include the three-part layer but a particle is coated with the three-part composite layer described herein.

In preferred embodiments, cells that produce a therapeutic substance, e.g., islet cells, are disposed in an internal particle and a capture agent, e.g., an antibody, is disposed on the internal particle or in the super matrix.

In preferred embodiments, the internal particle matrix hinders the passage, and preferably essentially completely prevents the passage of: molecules having a molecular weight of more than about 400 kDa, preferably more than about 150 kDa, preferably more than about 100 kDa, and most preferably more than about 50 kDa; or of immune system components such as Ig molecules or complement; or host-derived cells.

In preferred embodiments, a capture agent is disposed on the surface of the internal particle matrix.

In preferred embodiments, the internal particle coating is or includes: a monodisperse polyamino acid, e.g., monodisperse polylysine (PLL) or monodisperse PLO. A particularly preferred coating is monodisperse polyamino acid. e.g., monodisperse polylysine or monodisperse polyomithine, having a molecular weight of less than 15 kDa, more preferably of less than 10 kDa, more preferably of less than 5 kDa. Particularly preferred are monodisperse polyamino acids, e.g., monodisperse polylysines, with a molecular weight of about 1 kDa-4 kDa, about 1 kDa-less than 4 kDa. e.g., 3.7 kDa, or about 5 kDa to less than 15 kDa, or about 5 kDa to less than about 10 kDa, e.g., 9 kDa-10 kDa, e.g., 9.7 kDa. Also preferred are monodisperse polyamino acid, e.g., PLL or PLO, coatings in the range of 1 or 2-10 kDa, preferably in the range of 1-2, 1-3, or 1-4 kDa. Preferred coatings are volume-reducing coatings.

In preferred embodiments, the internal particle coating hinders the passage, and preferably essentially completely prevent the passage of: molecules having a molecular weight of more than about 400 kDa, preferably more than about 150 kDa, preferably more than about 100 kDa, and most preferably more than about 50 kDa; or of immune system components such as Ig molecules or complement; or host-derived cells.

In preferred embodiments, the diameter of the internal particle, before application of a volume-reducing coating, between 50 and 700 microns, more preferably between 100 and 500 microns, more preferably between 200 and 400 microns, and most preferably about 300 microns in diameter. The diameter of the internal particles, after application of a volume-reducing coating, is preferably between 35 and 500 microns, more preferably between 75 and 400 microns, more preferably between 100 and 300 microns, and most preferably about 200 microns in diameter.

In preferred embodiments, the particle matrix is or includes: a gel, e.g., a hydrogel, e.g., an alginate or agarose gel. In preferred embodiments the particle matrix is other than a liquid. The particle matrix can include substances which impede the passage of recipient-derived molecules or cells, e.g., it can include polyethylene oxide (PEO), polystyrene sulfonic acid (PSA), polyethylene glycol (PEG), or polyornithine (PLO).

In preferred embodiments, a capture agent is disposed within the particle matrix.

In preferred embodiments, the particle matrix hinders the passage, and preferably essentially completely prevent the passage of: molecules having a molecular weight of more than about 50 kDa, preferably more than about 100 kDa, preferably more than about 150 kDa, and most preferably more than about 400 kDa; or of immune system components such as Ig molecules or complement; or recipient-derived cells.

In preferred embodiments, the particle coating is or includes: a monodisperse polyamino acid, e.g., monodisperse polylysine (PLL) or PLO. A particularly preferred coating is monodisperse polyamino acid, e.g., monodisperse polylysine or monodisperse polyornithine, having a molecular weight of less than 15 kDa, more preferably of less than 10 kDa, more preferably of less than. 5 kDa. Particularly preferred are monodisperse polyamino acids, e.g., monodisperse polylysines, with a molecular weight of about 1 kDa-4 kDa (or about 1 kDa-less than 4 kDa) e.g., 3.7 kDa, or about 5 kDa to less than 15 kDa, or about 5 kDa to less than about 10 kDa, e.g., 9 kDa-10 kDa, e.g., 9.7 kDa. Also preferred are monodisperse polyamino acid, e.g., PLL or PLO, coatings in the range of 1 or 2-10 kDa, preferably in the range of 1-2, 1-3, or 1-4 kDa. Preferred coatings are volume-reducing coatings.

In preferred embodiments, the particle coating hinders the passage, and preferably essentially completely prevent the passage of: molecules having a molecular weight of more than about 50 kDa, preferably more than about 100 kDa, preferably more than about 150 kDa, and most preferably more than about 400 kDa; or of immune system components such as Ig molecules or complement; or recipient-derived cells.

In preferred embodiments, the diameter of the particle, before application of a volume-reducing coating, is between 200 and 1,000 microns, more preferably between 400 and 800 microns, more preferably between 500 and 700 microns, and most preferably about 600 microns in diameter. The diameter of the particles, after application of a volume-reducing coating, is preferably between 100 and 700 microns, more preferably between 250 and 550 microns, more preferably between 300 and 500 microns, and most preferably about 400 microns in diameter.

In preferred embodiments, the super matrix has little or no ability to exclude low molecular weight species, e.g., IgG or complement, with this property being relegated to other components of the microcapsule.

In preferred embodiments a capture agent is disposed on the surface of the super matrix.

In preferred embodiments, the gel super matrix is or includes: a hydrogel, e.g., an alginate or agarose gel. In preferred embodiments the super matrix is other than a liquid. The super matrix can include substances which impede the passage of recipient-derived molecules or cells, e.g., it can include polyethylene oxide (PEO), polystyrene sulfonic acid (PSA), polyethylene glycol (PEG), or polyomithine (PLO). A high molecular weight, molecule, e.g., a polymer, e.g., PEO, with a molecular weight of 1-8 million daltons, or more, can be added to the super matrix to confer immunoisolating properties.

In preferred embodiments, the super matrix hinders the passage, and preferably essentially completely prevent the passage of: molecules having a molecular weight of more than about 50 kDa, preferably more than about 100 kDa, preferably more than about 150 kDa, and most preferably more than about 400 kDa; or of immune system components such as Ig molecules or complement; or recipient-derived cells.

In preferred embodiments the super matrix has little or no ability to exclude low molecular weight species, e.g., IgG or complement, with this property being relegated to other components of the microcapsule.

In preferred embodiments, the super matrix coating (which is optional) is or includes: a monodisperse polyamino acid, e.g., monodisperse polylysine (PLL) or monodisperse PLO. Particularly preferred coating is monodisperse polyamino acid. e.g., monodisperse polylysine or monodisperse polyornithine, having a molecular weight of less than 15 kDa, more preferably of less than 10 kDa, more preferably of less than 5 kDa. Particularly preferred are monodisperse polyamino acids, e.g., monodisperse polylysines, with a molecular weight of about 1 kDa-4 kDa (or about 1 kDa-less than 4 kDa) e.g., 3.7 kDa, or about 5 kDa to less than 15 kDa, or about 5 kDa to less than about 10 kDa, e.g., 9 kDa-10 kDa, e.g., 9.7 kDa. Also preferred are monodisperse polyamino acid, e.g., monodisperse PLL or monodisperse PLO, coatings in the range of 1 or 2-10 kDa, preferably in the range of 1-2, 1-3, or 1-4 kDa.

In preferred embodiments, the particle coating hinders the passage, and preferably essentially completely prevent the passage of: molecules having a molecular weight of more than about 400 kDa, preferably more than about 150 kDa, preferably more than about 100 kDa, and most preferably more than about 50 kDa; or of immune system components such as Ig molecules or complement; or recipient-derived cells.

In preferred embodiments, the diameter of the double composite microreactor is, before application of a volume-reducing coating, between 400 and 1,500 microns, more preferably between 500 and 1,300 microns, more preferably between 600 and 1,100 microns, and most preferably about 900 microns in diameter. The diameter of the double composite microreactor is, after application of a volume-reducing coating, is preferably between 300 and 1,300 microns, more preferably between 400 and 1,200 microns, more preferably between 500 and 1,000 microns, and most preferably about 800 microns in diameter.

In preferred embodiments, the composite microreactor includes a plurality of internal particles, e.g., between 2 and 100, e.g., between 2 and 10, internal particles.

In preferred embodiments, the composite microreactor includes a plurality of particles, e.g., between 2 and 100, e.g., 2 and 10, particles.

In preferred embodiments, the double composite microreactor is a component of a higher order composite, e.g., a third or fourth order composite.

In a preferred embodiment, one or more components of the composite is geometrically stabilized, as is taught in PCT/US96/03135 and U.S. patent application Ser. No. 08/402,209, Filed Mar. 10, 1995. For example: the first internal particle matrix is geometrically stabilized, e.g., by allowing it to age for between 2 hours and 14 days, e.g., for between 12 hours and 4 or 5 days, prior to coating it; the first internal particle is geometrically stabilized, e.g., by allowing it to age for between 2 hours and 14 days, e.g., for between 12 hours and 4 or 5 days prior to embedding it in the particle matrix; the first particle matrix is geometrically stabilized, e.g., by allowing it to age for between 2 hours and 14 days, e.g., for between 12 hours and 4 or 5 days, prior to coating it; the first particle is geometrically stabilized, e.g., by allowing it to age for between 2 hours and 14 days, e.g., for between 12 hours and 4 or 5 days prior to embedding it in the super matrix; the super matrix is geometrically stabilized, e.g., by allowing it to age for between 2 hours and 14 days, e.g., for between 12 hours and 4 or 5 days prior to coating it; the coated super matrix is geometrically stabilized, e.g., by allowing it to age for between 2 hours and 14 days, e.g., for between 12 hours and 4 or 5 days.

In preferred embodiments, the internal particle coating has a lower molecular weight exclusion number than does the particle matrix, the super matrix, the outer coating (if present), or a combination of one or more of these, e.g., the internal particle coating excludes recipient immune molecules, e.g., IgG or complement, and the particle matrix, super matrix, the outer coating (if present), or a combination of these, allows immune molecules, e.g., IgG, or complement, to pass but excludes the passage of recipient cells.

In preferred embodiments, the outer surface of the composite is biologically compatible, e.g., it is sufficiently smooth that it inhibits fibrotic encapsulation of the composite; the outer surface of the composite is biologically compatible, e.g., it is sufficiently smooth that it inhibit fibrotic encapsulation of the composite but the surface of the internal particle (or of the particle) is not biologically compatible, e.g., it is not sufficiently smooth to inhibit fibrotic encapsulation.

In preferred embodiments: the first source is an islet; the second source is an islet; the third source is an islet; the fourth source is an islet; one source is an islet and another source is other than an islet, e.g. an erythrocyte, an acinar cell, or an adrenal cell.

In preferred embodiments: an internal particle coating is a low molecular weight monodisperse polyamino acid e.g., 1 kDa-4 kDa, about 1 kDa-less than 4 kDa and a particle coating is a monodisperse low molecular weight polyamino acid e.g., 5 kDa to less than about 10 kDa, 5 kDa to less than about 15 kDa, e.g., about 9 kDa-10 kDa. Also preferred are monodisperse polyamino acid, e.g., monodisperse PLL or monodisperse PLO, coatings in the range of 1 or 2-10 kDa, preferably in the range of 1-2, 1-3, or 1-4 kDa.

In preferred embodiments: an internal particle coating has an exclusion limit of about 150 kDa and the first particle coating has an exclusion limit of about 400 kDa.

In preferred embodiments: an internal particle coating has an exclusion limit of about 150 kDa and the particle matrix, first particle coating, super matrix, outer coating, or a combination thereof, has an exclusion limit of about 400 kDa.

In preferred embodiments: an internal particle coating has an exclusion limit which is lower than that of the particle coating, e.g., the internal particle coating has an exclusion limit which will exclude molecules which are the size of IgG, or Clq, or larger, and the particle coating has an exclusion limit which, though permeable to IgG, or Clq, will exclude objects, e.g., cells, having a molecular weight of one million or more.

In preferred embodiments: an internal particle coating has an exclusion limit which is lower than that of the particle coating, e.g., the internal particle coating has an exclusion limit which will exclude molecules which are the size of IgG, or Clq, or larger, and the particle matrix, first particle coating, super matrix, outer coating, or a combination thereof, has an exclusion limit which, though permeable to IgG, or Clq, will exclude objects, e.g., cells, having a molecular weight of one million or more.

In preferred embodiments: there is a buffer-zone component, e.g., the particle matrix, which is disposed between a component which is not biocompatible, e.g., which is not anti fibrotic, e.g., the internal particle coating, and a component which has an exclusion limit which excludes the passage of recipient cells, e.g., the super matrix or outer coating.

In preferred embodiments: an internal particle coating has an exclusion limit which will exclude molecules which are the size of IgG, or Clq, or larger, the particle matrix is not free of defects which arise from the use of non-geometrically stabilized components, and the super matrix, outer coating, or a combination thereof, has an exclusion limit which, though permeable to IgG, or Clq, will exclude objects, e.g., cells, having a molecular weight of one million or more.

In preferred embodiments, the first particle of the double composite microreactor further includes:

one, or a plurality, of a second internal particle which includes:

(i) a second source of a therapeutic substance, e.g., an islet;

(ii) a second internal particle matrix which contacts the second source;

(iii) a second internal particle coating of a monodisperse polyamino acid enclosing the second internal particle matrix;

In preferred embodiments, the double composite microreactor further includes:

one, or a plurality, of a second particle which includes:

(a) a third internal particle which includes:

(i) a third source of a therapeutic substance, e.g., an islet,

(ii) a third internal particle matrix which contacts the third source,

(iii) (optionally) a third internal particle coating of a monodisperse polyamino acid enclosing the third internal particle matrix; and

(b) (optionally) a fourth internal particle which includes:

(i) a fourth source of a therapeutic substance, e.g., an islet,

(ii) a fourth internal particle matrix which contacts the fourth source,

(iii) a fourth internal particle coating of a monodisperse polyamino acid enclosing the fourth internal particle matrix.

In preferred embodiments: one or more of the particle matrix, particle covering, or super matrix, prevents contact of host cells capable of fibrotic reactions with the internal particle coating; the particle matrix, super matrix or the outer coating (if present) is free of defects which arise from the inclusion of non-geometrically stabilized components, e.g., non-geometrically-stabilized internal particles; at least two, or three, or four, components chosen from the group of the particle matrix, the particle coating, the super matrix, and the outer coating (if present), provides a molecular weight cutoff that prevents molecules larger than about 150,000 daltons from coming into contact with the internal particle coating; the internal particle molecular weight cutoff is provided by a pore structure of the internal particle matrix, and that pore structure results, e.g., from cross-linking of the internal particle gel; the molecular weight cutoff the super matrix is provided by a pore structure of the super matrix.

Preferred embodiments lack an outer coating.

In preferred embodiments, the outer surface of the double (or higher order) composite microreactor is a gel, e.g., an alginate gel. In more preferred embodiments the outer surface of the composite microreactor is a gel, e.g., an alginate gel, the outer surface of which has been modified, e.g., by cross-linking, to produce a covalently modified gel surface, e.g., to form a coating.

In preferred embodiments, the internal particle coating is monodisperse polylysine of about 5 kDa to less than about 15 kDa, or about 5 kDa to less than about 10 kDa, e.g., 9 kDa-10 kDa, and the particle coating is monodisperse polylysine of about 5 kDa to less than about 15 kDa, or about 5 kDa to less than about 10 kDa, e.g., about 9 kDa-10 kDa. Also preferred are monodisperse polyamino acid, e.g., monodisperse PLL or monodisperse PLO, coatings in the range of 1 or 2-10 kDa, preferably in the range of 1-2,1-3, or 1-4 kDa.

In preferred embodiments the internal particle coating is monodisperse polylysine of about 1 kDa-4 kDa (or about 1 kDa-less than 4 kDa) e.g., about 2 kDa-3 kDa, and the particle coating is monodisperse polylysine of about 5 kDa to less than about 15 kDa, or about 5 kDa to less than about 10 kDa, e.g., about 9 kDa-10 kDa. Also preferred are monodisperse polyamino acid, e.g., monodisperse PLL or monodisperse PLO, coatings in the range of 1 or 2-10 kDa, preferably in the range of 1-2, 1-3, or 1-4 kDa.

In preferred embodiments, the internal particle coating is monodisperse polylysine of about 1 kDa-4 kDa (or about 1 kDa-less than 4 kDa) e.g., about 2 kDa-3 kDa, and the particle coating is monodisperse polylysine of about 1 kDa-4 kDa (or about 1 kDa-less than 4 kDa) e.g., about 2 kDa-3 kDa. Also preferred are monodisperse polyamino acid, e.g., monodisperse PLL or monodisperse PLO, coatings in the range of 1 or 2-10 kDa, preferably in the range of 1-2, 1-3, or 14 kDa.

In preferred embodiments the outer component of the double composite microreactor, i.e., the component in contact with the recipient, is at least 50, 75, 90, 95, 97, or 98%, water.

In preferred embodiments, one or more components of the double composite microreactor is of sufficient diameter, or of sufficient thickness, such that it imposes a substantial distance (or separation) between recipient cells, e.g., lymphocytes, macrophages, or NK cells, and the source of a therapeutic substance. In more preferred embodiments the thickness (e.g., the distance between its inner surface and its outer surface) of a component, e.g., a matrix, e.g., a particle matrix or super matrix, is: at least 5, 10, 20, 50, 75, 100, or 200 microns. In more preferred embodiments the distance between recipient cells and the source of a therapeutic substance is: at least 5, 10, 20, 50, 75, 100, or 200 microns; sufficient such that exposure of the source of a therapeutic substance to small molecules (e.g., molecules which are not excluded by a component which excludes IgG, e.g., cytokines, nitric oxide (NO), and other toxic moieties) released by recipient cells is substantially reduced (e.g., by diffusion), e.g., reduced at least 10, 20, 50, 75, or 90%; sufficient such that the concentration of small molecules (e.g., molecules which are not excluded by the selectively permeable barrier-components of the double composite microreactor, e.g., cytokines, NO, and other toxic moieties) released by recipient cells is substantially reduced (e.g., by diffusion), e.g., reduced at least 10, 20, 50, 75, or 90% at the source of a therapeutic substance. In more preferred embodiments: the distance is supplied by one or both of the particle matrix and the super matrix.

In preferred embodiments, the distance between the source of the therapeutic substance and recipient [host] cells is sufficient that a capture agent with affinity for antigens found on source cells do not affect the viability of source cells; e.g., antigen release is reduced at least 20%, 50%, 75%, 90%, most preferably 95%, while initial source cell viability remains at least 25%, 70%, 75%, 85%, 95% of initial viability level.

In preferred embodiments, one or more components of the double composite microreactor is of sufficient diameter, or of sufficient thickness, such that it imposes a substantial distance (or separation) between recipient cells, e.g., lymphocytes, macrophages, or NK cells, and one or both of the source of a therapeutic substance or donor antigen (other than the therapeutic substance) released by the source (e.g., donor proteins, which could stimulate a recipient response against donor tissue). In more preferred embodiments the thickness (e.g., the distance between its inner surface and its outer surface) of a component, e.g., a matrix, e.g., a particle matrix or super matrix, is: at least 5, 10, 20, 50, 75, 100, or 200 microns. In more preferred embodiments the distance between recipient cells and the source of a therapeutic substance is: at least 5, 10, 20, 50, 75, 100, or 200 microns; sufficient such that the amount, number, or concentration of a donor antigen, released into the recipient, or contacting recipient cells, is substantially reduced (e.g., by diffusion, or by trapping in or exclusion by the component or components which supply the separation), e.g., reduced by at least 10, 20, 50, 75, or 90%; sufficient such that contact of cells of the recipient with donor antigens, e.g., proteins, which protrude from or extend through the internal particle matrix or internal particle coating, or both, is substantially reduced (e.g., by diffusion, or by trapping in with a capture agent or by exclusion by the component or components which supply the separation), e.g., reduced by at least 10, 20, 50, 75, or 90%; sufficient to inhibit acute release of donor antigens. In more preferred embodiments: the distance or separation is supplied by one or both of the particle matrix and the super matrix.

As used herein, a source of a therapeutic substance can include a composition of matter which produces or releases a therapeutic substance, e.g., a protein, e.g., an enzyme, hormone, antibody, or cytokine, a sense or anti-sense nucleic acid, e.g., DNA or RNA, or other substance which can exert a desired effect on a recipient. The therapeutic substance can also be a composition of matter which absorbs or modifies or detoxifies a substance produced by the recipient. The source of a therapeutic substance can be a tissue or a living cell; a eukaryotic cell, e.g., a rodent, canine, porcine, or human cell; a prokaryotic cell, e.g., a bacterial cell; a fungal or plant cell; a cell which is genetically engineered, e.g., a cell which is genetically engineered to produce a protein, e.g., a human protein. The source of a therapeutic substance can be or include an autologous, an allogeneic, or a xenogeneic cell. For example, the cell is: an autologous cell, i.e., a cell which is taken from the individual recipient into which the cell will be implanted; an allogeneic cell, i.e., a cell which is taken from a different individual of the same species as the recipient into which the cell will be implanted; a xenogeneic cell, i.e., a cell which is from a different species than the recipient into which the cell will be implanted. In the case of an allogeneic cell, the cell can be fully matched or partially matched for MHC class I loci, fully matched or partially matched for MHC class II loci, and fully matched or partially matched for minor loci.

Embodiments of the invention feature the use of coatings, which, result in a decrease in the volume of the coated particle, as is described in PCT/US96/03135 and U.S. patent application Ser. No. 08/402,209, Filed Mar. 10, 1995.

The number of host molecules that can invade a component of a composite microreactor decreases substantially in the inward direction. As is discussed herein, the inner most member of a double composite microreactor is an internal particle. An internal particle is embedded in the next most outermost component, the particle matrix. The particle is in turn embedded in the next most outer component, the supermatrix.

Of source antigens other than the therapeutic substance released from the microreactor, only a small proportion will enter a particle matrix, and only a small proportion of those that enter a particle matrix will be able to be released from the microreactor.

Pretreatment of Microreactors

As discussed herein, the therapeutic source, e.g., implanted tissue, e.g., islet cells or other pancreatic tissue, can release antigens which can escape from an implantable device and elicit a host immune response. The number of antigens is, in many cases, greater when the cells are first encapsulated than over time. Thus, a microreactor or a component thereof, which contains a source of a therapeutic substance can be aged or incubated for a period of time sufficient to permit release or shedding of antigens prior to incorporating a capture agent into the microreactor. The incubation period allows for the release of antigen prior to inclusion of the capture agent thereby preventing saturation or occupation of capture agents and increasing the useful lifetime of the microreactor. The implantable device, or a component thereof containing the therapeutic source, can be assembled and aged for a sufficient period of time to allow release of antigen, for example, for at least 10, 20, 30, 40 or. 50 days. In the case of a composite microreactor wherein the source is located in the internal particle, the internal particle can be manufactured and aged for a sufficient amount of time and then incorporated into a matrix in which the capture agent is placed.

Implantation

Implantable devices can be implanted by methods known to those skilled in the art. The implantable devices can be implanted into a host by injection with a standard catheter or syringe, e.g., with a 16 gauge needle for beads less than 1000 μm in diameter. Larger implantable devices can be inserted via a small incision, e.g., with a catheter or funnel-like device. The beads are preferably implanted into the host intraperitoneally. The beads can also be implanted intramuscularly or subcutaneously. Alternatively, the beads can also be implanted into immunoprivileged sites such as the brain, testes, or thymus, where the host's immune response is least vigorous, as described in Chapter 7 of Lanza et al. (eds.), Immunomodulation of Pancreatic Islets (RG Landes, Texas, 1994). Composite implantable devices can also be introduced at a site where the substance provided by the composite implantable device is needed locally. E.g., an implantable device that provides □-interferon could be implanted in tumors. The implantable devices of the invention can be delivered to a subcutaneous site. The composite implantable devices can be inserted through a small surgically created opening using a gun/trocar type device that slips the beads under the skin.

A suitable host for the invention can be a subject or a patient, sometimes referred to as “recipient.” The term “subject,” as used herein, refers to a living animal or human in need of therapy for, or susceptible to, a condition, which is remediable through implantable device implantation and reduction of potential host immune rejection of the implantable device. In preferred embodiments, the subject is a mammal, including humans and non-human mammals such as dogs, cats, pigs, cows, sheep, goats, horses, rats, and mice. In the most preferred embodiment, the subject is a human. The term “subject” does not preclude individuals that are normal in all respects. The subject can be a candidate for future treatment by microcapsular implantation, formerly have been treated surgically or by chemotherapy, and can be under treatment by microcapsular implantation, and can have been so treated in the past.

The term “patient,” as used herein, refers to a human subject who has presented at a clinical setting with a particular symptom or symptoms suggesting treatment by implantation of implantable devices and suppression of the immune response concomitant to or subsequent to implantation. A patient's diagnosis can alter during the course of disease progression, such as development of further disease symptoms, or remission of the disease, either spontaneously or during the course of a therapeutic regimen or treatment.

The invention will now be described further by way of the following examples.

### EXAMPLES

A number of well-characterized disorders caused by the loss or malfunction of specific cells in the body are amenable to implantable device-medicated replacement therapy. For example, in addition to the islets of Langerhans, which can be used for the treatment of diabetes as described above, hepatocytes can be used for the treatment of hepatic failure, adrenal gland cells can be used for the treatment of Parkinson's disease, nerve growth factor (NGF)-producing cells can be used for the treatment of Alzheimer's disease, factors VIII- and IX-producing cells can be used for the treatment of hemophilia, and endocrine cells can be used for the treatment of disorders resulting from hormone deficiency, e.g., hypoparathyroidism.

Moreover, by using recombinant DNA methods to supply a cell which produces a disease product, or encapsulating other tissues, implantable devices can be used to treat patients suffering from chronic pain, cancer (e.g., hairy cell leukemia, melanoma, and renal carcinoma), AIDS (treated by immunological augmentation), Kaposi's Sarcoma (treated by administration of interferon, IL-2, or TNF-α), primary hematologic disorders, patients with long-lasting aplasia, and patients who are myelosuppressed (treated by bone marrow transplantation and aggressive chemotherapy). Implantable devices should also be useful in the treatment of affective disorders, e.g., Huntington's Disease, Duchenne's Muscular Dystrophy, epilepsy, infertility. Implantable devices can also be used to promote wound healing and to treat traumatic, mechanical, chemical, or thermal injuries, e.g., spinal cord injuries, and in wound healing.

Implantation of specific cells can also serve to detoxify, modify, or remove substances from the circulation, e.g., drugs, poisons, or toxins. For example, the implantation of appropriate living cells restores normal physiologic function by providing replacement for the diseased cells, tissues, or organs, e.g., in hepatic encephalopathy (produced by liver disease) or uremia (produced by kidney failure).

In embodiments of the invention, the encapsulated cells can release fairly large molecules, e.g., IgG molecules. In many applications the critical host component which must be excluded is Clq, which has a molecular weight of about 410 kDa. Thus, the molecular weight cutoff will be about 400 kDa and molecules of up to this size can be released. Genetically engineered cells can also be used in the methods of the invention. For example, cells can be engineered to release larger products, e.g., IgG.

In each application, a sufficient number of composite implantable devices, containing the desired living cells, can be implanted into the patient, e.g., surgically or with a syringe. The implantable devices are implanted, e.g., intraperitoneally, for a systemic effect, or into a particular location, e.g., the brain to treat Parkinson's disease, or the spinal cord to chronic pain or treat spinal cord injuries, for a local effect.

The dose of implantable devices to be used is determined initially from results of in vitro studies. In addition, in vivo results in, e.g., mice, rats, or dogs will facilitate more accurate assessment of required doses, as these tests are generally predictive of efficacy in human patients. For example, canine insulin dependent diabetes represents an excellent model of cellular and humoral autoimmunity (Nelson, Diabetes Spectrum 5:324-371 (1992))

The implantable devices are intended to remain in the patient with viable donor cells for extended periods of time up to several months or years. However, if it is determined that the donor cells are no longer viable, e.g., by monitoring the patient's blood for a certain level of the protein secreted by the donor cells, it is a simple task to remove the implantable devices and renew the supply of beads in the patient.

Diabetes Mellitus

To treat diabetes, e.g., in a dog or human patient, the implantable beads preferably encapsulate isolated canine or porcine islets or other cells that produce insulin or insulin-like growth factor 1 (IGF-1). Islets are prepared and encapsulated using procedures described above. Insulin secretory activity of the encapsulated cells or islets is determined both in static culture, e.g., expressed per islet volume, and based on the capability of the islets to respond to graded concentrations of glucose. These values are established as described above. Once the insulin secretion activity of a particular batch of encapsulated islets is determined, the proper number of beads can be determined and implanted into a diabetic patient. For example, to treat a human patient that requires 20 to 50 units of insulin per day, the total number of beads should be selected to contain a total of about 1.0 to 2.5 million porcine islets. For beads designed to contain, on average, 30,000 islets/ml of gel, the proper dosage would be beads made from 30 to 85 ml of gel.

Hemophilia

Hemophilia is an X-linked hereditary bleeding disorder caused by Factor VIII or Factor IX deficiency. Recombinant methods have now been successfully used to create Factor VIII- and Factor IX-producing cells as described above. Encapsulation in implantable devices and implantation of such cells according to the present invention can thus be used for an improved treatment for hemophilia.

Hepatic Diseases

Hepatocyte transplantation is useful not only for irreversible hepatic failure, but for several disease processes including hereditary enzyme abnormalities, acute hepatic failure, where the ability of the liver to regenerate can occur, and as a bridge to whole liver transplantation in patients who develop sudden hepatic failure, either because of medical progression or because of rejection-related complications.

Wong and Chang, Biomat. Art. Cells Art. Org., 16:731 (1988), have demonstrated the viability and regeneration of microencapsulated rat hepatocytes implanted into mice. Viable hepatocytes were microencapsulated in alginate-poly-(L-lysine) and implanted intraperitoneally into normal and galactosamine-induced liver failure mice. Eight days after implantation in the mice with induced liver failure, the viability of the encapsulated rat hepatocytes increased from 42% to nearly 100%. After 29 days, the viability of the encapsulated hepatocytes implanted in normal mice also increased from 42% to nearly 100%. By contrast, free rat hepatocytes implanted into mice all died within four or five days after xenotransplantation. Implantable devices are well-suited to treat hepatic failure.

Other investigators have shown that microencapsulated hepatocytes continue the synthesis and secretion of many specific proteins and enzymes. Cai et al., Hepatology, 10:855 (1989), developed and evaluated a system of microencapsulation of primary rat hepatocytes. Urea formation, prothrombin and cholinesterase activity, the incorporation of titrated leucine into intracellular proteins, and the immunolocation of synthesized albumin were monitored in culture. Despite gradual decreases in some of these activities, the encapsulated hepatocytes continued to function throughout the 35-day observation period. In addition, Bruni and Chang, Biomat. Art. Cells Art. Org., 17:403 (1989), demonstrated the use of microencapsulated hepatocytes to lower bilirubin levels in hyperbilirubinemia. Microencapsulated hepatocytes were injected into the peritoneal cavity of Grunn rats. Bilirubin dropped from 14 milligrams/100 ml to 6 milligrams/100 ml, and remained depressed after 90 days. Again, implantable devices can be used as described above to treat these hepatic diseases.

Parkinson's Disease

Parkinson's disease is a neuronal system disease, involving a degeneration of the nigrostriatal dopaminergic system. Experimental work in both rodents and nonhuman primates has shown that transplantation of fetal tissue containing substantia nigra (dopaminergic) neurons from ventral mesencephalon to dopamine-depleted striatum reinstates near-normal dopamine interinnervation and reduces motor abnormalities. In addition, implantation of adrenal chromaffin cells has been shown to reverse chemically-induced Parkinson's disease in rodents.

Widnere-t al., Transplant. Proc., 23:793 (1991), reported evidence of fetal nigral allograft survival and function up to 10 months after transplantation and immunosuppression (cyclosporin, azathioprine, and prednisone) in a human Parkinson's patient. Beginning from the second month after the transplantation, they observed a progressive decrease in limb rigidity, increased movement speed in a number of arm, hand, and foot movements, and prolonged “on” periods (greater than 80% increase) after a single dose of L-dopa.

Thus, transplantation of fetal neural tissue, or cells genetically engineered to produce dopamine and nerve growth factors or other neurotropic factors, should have a great potential as a new therapeutic approach in patients with neurological disorders. However, in the case of transplanted xenogeneic donor tissue, rejection would pose a serious problem, even by the combined approach of using an immunoprivileged site and by employing immunosuppressive drugs. Therefore methods of the invention permit a novel approach to this problem, i.e., the delivery of dopamine for the treatment of Parkinson's disease using encapsulated donor tissue harvested from animals or genetically engineered cells.

Alzheimer's Disease

An estimated 2.5 to 3.0 million Americans are afflicted with Alzheimer's disease. The disease is characterized by a progressive loss of cognitive function associated with degeneration of basal forebrain cholinergic neurons. Studies in animals indicate that Nerve Growth Factor (NGF), e.g., brain-derived neurotrophic factor (BDNF) and neurotrophin-3 (NT-3), available from Regeneron and Amgen, respectively, and other neurotropic factors normally act to support the viability and function of these neuron cells, and that continuous infusion of NGF into the ventricles can prevent injury-induced degeneration of cholinergic neurons as described in Williams et al., P.N.A.S., USA, 83:9231 (1986). This treatment correlates with improved cognitive function in rodents with memory impairment as described in Fisher et al., Neurobiol. Aging, 10:89 (1989).

These studies suggest that implantable devices containing grafts of recombinant or natural NGF-secreting tissue such as astroglial cells or developing skin, can be used to treat patients suffering from Alzheimer's disease.

Gene Therapy

Gene therapy is an approach to treating a broad range of diseases by delivering therapeutic genes directly into the human body. Diseases that can potentially be cured by gene therapy include diseases associated with the aging population such as cancer, heart disease, Alzheimer's disease, high blood pressure, atherosclerosis and arthritis; viral infectious diseases such as acquired immune deficiency syndrome (AIDS) and herpes; and inherited diseases such as diabetes, hemophilia, cystic fibrosis, and muscular dystrophy.

In one particular example, a favored approach for human gene therapy involves the transplantation of genetically-altered cells into patients, e.g., as described Rosenberg, et al., New Eng. J. Med., 323:570-578 (1988). This approach requires the surgical removal of cells from each patient to isolate target cells from nontarget cells. Genes are introduced into these cells via viral vectors or other means, followed by transplantation of the genetically-altered cells back into the patient. Although this approach is useful for purposes such as enzyme replacement therapy (for example, for transplantation into a patient of cells that secrete a hormone that diseased cells can no longer secrete), transplantation strategies are less likely to be suitable for treating diseases such as cystic fibrosis or cancer, where the diseased cells themselves must be corrected. Other problems commonly encountered with this approach include technical problems, including inefficient transduction of stem cells, low expression of the transgene, and growth of cells in tissue culture which can select for cells that are predisposed to cancer.

The methods of the invention are well suited to avoid these problems, because they allow the use of standard human cell lines of, e.g., fibroblast cells, epithelial cells such as HeLa cells, and hepatoma cells such as HepG2, as the implanted cells, rather than requiring the surgical removal of cells from the patient. These cell lines are genetically altered as required by standard techniques and are encapsulated and implanted into the patient. These cell lines are much easier to obtain, culture, and work with than individual patients' cells. Moreover, since the implantable devices prevent the patient's immune system from recognizing and attacking the implanted cells, any human cell lines can be used, making the technique of gene therapy more universally applicable.

Hypoparathyroidism

Acute and chronic symptoms of hypoparathyroidism result from untreated hypocalcemia, and are shared by both hereditary and acquired hypoparathyroidism. The hereditary form typically occurs as an isolated entity without other endocrine or dermatologic manifestations or, more typically, in association with other abnormalities such as defective development of the thymus or failure of other endocrine organs such as the thyroid or ovary. Acquired hypoparathyroidism is usually the result of inadvertent surgical removal of all the parathyroid glands, and is a problem in patients undergoing operations secondary to parathyroid adenoma or hyperplasia. Hypoparathyroidism has been treated in hypocalcemic rats by the administration of microencapsulated parathyroid cells that served as a bioartificial parathyroid. Parathyroid cells can also be encapsulated in implantable devices and used with the methods described herein in administration to animals and human patients.

Osteoporosis

The term osteoporosis covers diseases of diverse etiology that cause a reduction in the mass of bone per unit volume. These diseases can be treated by the administration of implantable devices containing cells that secrete insulin-like growth factor (IGF-1), estrogen in postmenopausal woman to reduce the negative calcium balance and decrease urinary hydroxyproline, androgens in the treatment of osteoporotic men with gonadal deficiency, or calcitonin for use in established osteoporosis.

Reproductive Disorders

There are numerous disorders of the ovary and female reproductive tract that can be treated with progestrogens, estrogens, and other hormones. These include progestrogen, e.g., progesterone, therapy to inhibit pituitary gonadotropins (precocious puberty in girls), and for prophylaxis to prevent hyperplasia in PCOD. Estrogen therapy is used in the treatment of gonadal failure, control of fertility, and in the management of dysfunctional uterine bleeding. Androgens, gonadotropins, and other hormones are used to treat disorders of the testis, e.g., androgen therapy in hypogonadal men, or gonadotropins to establish or restore fertility in patients with gonadotropin deficiency. Accordingly, these diseases can be treated with implantable devices containing the appropriate hormone-producing cells.

Huntington's Disease

Huntington's disease is characterized by a combination of choreoathetotic movements and progressive dementia usually beginning in midadult life. Distinctive for the disease is atrophy of the caudate nucleus and, to a lesser extent, other structures of the basal ganglia (putamen and globus pallidus). Rodent cells that secrete neurotropic factors have been implanted into the brains of baboons that have a condition similar to Huntington's disease and reversed some of the damaged nerve networks that, in Huntington's patients, lead to progressive loss of control over the body. Similarly, Huntington's disease in human patients can be treated by the administration of implantable devices that contain human or recombinant cells that secrete the appropriate neurotrophic factors.

Spinal Cord Injuries

The majority of spinal cord injuries result from damage to the surrounding vertebral column, from fracture, dislocation, or both. Treatment of such injuries involves the administration of nerve growth factors such as ciliary neurotropic factor (CNTF), insulin-like growth factor (IGF-1), and neurotropic factors, to enhance the repair of the central and peripheral nervous system. Thus, implantable devices containing cells that secrete such factors, either naturally or through genetic engineering, can be used to treat spinal cord injuries.

Mood (or Affective) Disorders

Mood disorders are a group of mental disorders such as schizophrenia characterized by extreme exaggerations and disturbances of mood and affect associated with physiologic (vegetative), cognitive, and psychomotor dysfunctions. Many mood disorders are associated with medical diseases that can be treated with implantable devices containing the appropriate cells such as hypothyroidism, Parkinson's disease, Alzheimer's disease, and malignancies as discussed herein. In addition, it has been shown that the neurotransmitter 5-hydroxyindol acetic acid (5-HIAA), a serotonin metabolite, is reduced in the cerebral spinal fluid of depressed patients. Deficits in other neurotransmitters such as dopamine and γ-aminobutyric acid (GABA) have also been identified in patients with major depression. Therefore, implantable devices containing cells that secrete these neurotransmitter are useful to treat these deficiencies.

Motor Neuron Diseases

Degenerative motor neuron diseases include ALS (see above), heritable motor neuron diseases such as spinal muscular atrophy (SMA), and those associated with other degenerative disorders such as olivopontocerebellar atrophies and peroneal muscular atrophy. These diseases can be treated by administration of implantable devices containing cells that secrete neurotropic factors like brain-derived neurotrophic factor (BDNF), and neurotrophin-3 (NT-3).

Acquired Immunodeficiency Syndrome (AIDS)

AIDS is caused by an underlying defect in cell-mediated immunity due to the human immunodeficiency virus (HIV), and causes persistent constitutional symptoms and/or diseases such as secondary infections, neoplasms, and neurologic disease. Patients can be treated to ameliorate symptoms by immunologic augmentation with implantable devices that contain cells genetically engineered to secrete, e.g., recombinant human IL-2 (to decrease suppressor cell activity resulting in an increased T cell adjuvant activity); or recombinant human INF-γ (macrophage augmentation). AIDS-related tumors such as Kaposi's sarcoma can be treated with encapsulated cells that secrete human interferon-α, interleukin-2 and tumor necrosis factor (TNF).

Amyotrophic Lateral Sclerosis (Lou Gehrig's Disease)

ALS is the most frequently encountered form of progressive motor neuron disease, and is characterized by progressive loss of motor neurons, both in the cerebral cortex and in the anterior horns of the spinal cord, together with their homologs in motor nuclei of the brainstem. ALS can be treated with implantable devices that contain cells that secrete nerve growth factors such as myotrophin, insulin-like growth factor (IGF-1), ciliary neurotropic factor (CNTF), brain-derived neurotrophic factor (BDNF) and neurotrophin-3 (NT-3). Animal studies with these factors (IGF-1 is available from Cephalon, CNTF from Regeneron, and NT-3 from Amgen), have demonstrated that they can stem the degenerative effects caused by nerve damage or disease.

Cancer

In most cases, cancer originates from a single stem cell which proliferates to form a clone of malignant cells. Growth is not properly regulated by the normal biochemical and physical influences in the environment. There is also a lack of normal, coordinated cell differentiation. Cancer cells develop the capacity for discontinuous growth and dissemination to other parts of the body.

Various cancers can be treated according to the invention by the administration of implantable devices containing cells that secrete interferon-α (IFN-α) (for solid tumors, hairy cell leukemia, Kaposi's sarcoma, osteosarcoma, and various lymphomas); recombinant interleukin-2 (IL-2) (for melanoma, renal carcinoma, and Kaposi's sarcoma); tumor necrosis factor (with IL-2 for Kaposi's sarcoma); recombinant human IFN-α and recombinant human colony stimulating factor-granulocyte macrophage (GM-CSF) (for Kaposi's sarcoma); recombinant human INF-γ (for macrophage augmentation); CSF (for aggressive chemotherapy, bone marrow transplantation, priming of leukemic cells to enhance sensitivity to chemotherapy and to support dose intensification); ciliary neurotropic factor (CNTF) and insulin-like growth factor (IGF-1) (for peripheral neuropathies caused by chemotherapy); adrenal gland cells (for pain relief when injected into the lower spine to secrete natural painkillers) and progesterone-producing cells (for palliation in endometrial and breast carcinoma).

Duchenne's Muscular Dystrophy

Duchenne's dystrophy is an X-linked recessive disorder characterized by progressive weakness of girdle muscles, inability to walk after age 12, kyphoscoliosis (curvature of the spine), and respiratory failure after the fourth decade. This disease can be treated by administration of implantable devices containing myoblast cells and growth factors. Myoblasts have been injected into young boys with Duchenne's muscular dystrophy to determine whether the cells can supply a structural protein that is missing. Researchers have observed muscle strength improvement in several of the boys.

Epilepsy

The epilepsies are a group of disorders characterized by chronic, recurrent, paroxysmal changes in neurologic function caused by abnormalities in the electrical activity of the brain. In some forms of focal epilepsy, inhibitory interneurons appear to be preferentially lost. Treatment with neurotropic factors and other neuropeptides such as has been found effective. Therefore, the implantable devices containing cells secreting these factors can be used to treat epilepsy.

Coating Alginate Microreactors Containing Pancreatic Islets with Polylysine

### Example 1

Islets were encapsulated in alginate and cultured in a culturing medium to form beads. The beads were then washed free of serum proteins introduced from the culturing medium.

Monodisperse polylysine (PLL) (Boehringer Mannheim) was added to a culture medium to produce a 0.4% polylysine (PLL) culture medium stock solution, which was preheated to 37° C. The beads were collected in a conical tube or flask and settled to determine bead volume. The beads were then washed three times with a volume of medium equal to 10× the bead volume. The beads were gently mixed for 3 minutes in a 0.2% PLL culture medium stock solution at 37° C. The coated beads were then removed from the PLL stock solution by filtering and washed 3× with culture medium medium equal to 2× the total coating volume, to remove excess PLL.

Permeability of Microsphere Sensors Coated with Monodisperse PLL Polymers

### Example 2

PLL Coating Procedure

Two 0.2% PLL coating solutions (in HEPES/saline buffer) were made from a 37° C. a 1% monodisperse PLL in HEPES/saline buffer stock solution. The first coating solution was 15× the microsphere bead volume. The second coating solution was 10× the bead volume. Both solutions were sterile filtered and kept at 37° C.

Microsphere sensor beads including a first fluorescent reagent component, Cy3.5-HSA (human serum albumin, molecular weight 68,000 g/mol) and a second fluorescent component Cy5.5-ConA (concanavalin A, molecular weight 102,000 g/mol), were coated with the first PLL coating solution for 5 minutes at 37° C. on a rocker. The beads were removed and rinsed three times with HEPES/saline buffer. The beads were then incubated for 60 minute at room temperature, while being protected from light. After 60 minutes the buffer was removed from the beads and the second PLL coating solution was added. The beads were coated for 5 minutes at 37° C. on a rocker. The beads were then removed and rinsed three times with HEPES/saline buffer.

Microsphere beads were coated with 0.2% PLL coating solutions as described above using monodisperse PLL samples having 33, 47, and 60 peptide residues. A permeability assay was performed on each set of PLL coatings. The beads were stored at 37° C. for three days and rinsed with buffer daily.

Leakage of the fluorescent components of the microsphere beads was measured on Day 0, 3, and 7 after the three day rinsing period. The amount of fluorescence in the solution surrounding the beads was measured using a fluorimeter and compared to the amount of fluorescence originally in the beads for each of the three coatings at the time points specified.

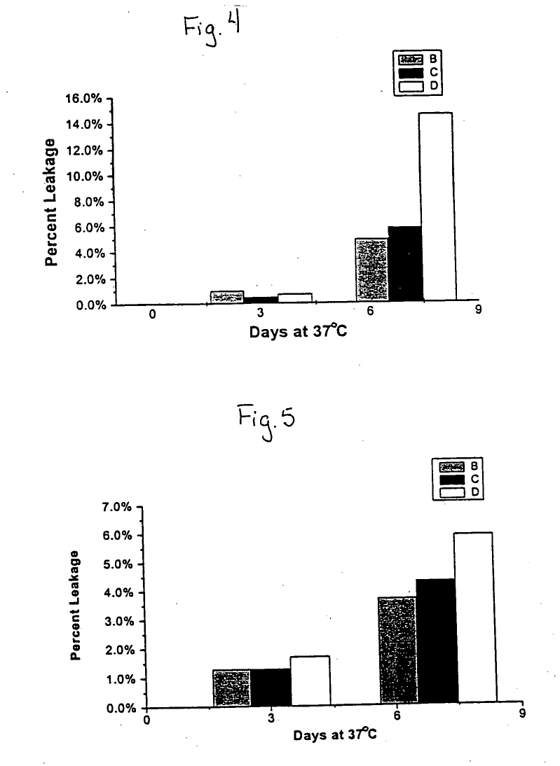

FIG. 4 shows the leakage of the Cy3.5-HSA fluorescent component from the above-coated microsphere beads, which were coated with 33, 47, and 60 peptide residue monodisperse PLL, indicated as B, C and D, respectively.

FIG. 5 shows the leakage of the Cy5.5-ConA fluorescent component from the above-coated microsphere beads, which were coated with the 33, 47, and 60 peptide residue monodisperse PLL, indicated as B, C and D, respectively.

Glucose Response of Microsphere Sensors Coated with 33 Residue Monodisperse PLL

### Example 3

Microsphere sensors were coated with 0.2% 33 residue monodisperse PLL coating solution and tested for glucose response in a fluorescence resonance energy transfer (FRET) assay.

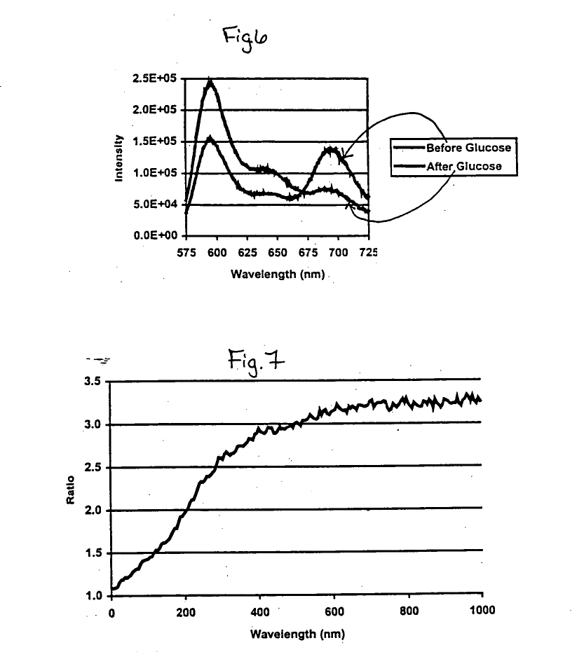

FIG. 6 shows the fluorescence spectra of the Cy3.5-HSA and Cy5.5-ConA components of the sensors before and after the addition of 500 mg/dL of glucose to the sensors in a spectrofluorimeter.

FIG. 7 shows the kinetic response of the same sensors to 500 mg/dL glucose.

It is to be understood that while the invention has been described in conjunction with the detailed description thereof, that the foregoing description is intended to illustrate and not limit the scope of the invention, which is defined by the scope of the appended claims. Other aspects, advantages, and modifications within the scope of the invention will be apparent to those skilled in the art to which the invention pertains.

All patents and publications referred to herein are hereby incorporated by reference

Other embodiments are within the claims.

What is claimed is:

1. An article comprising:
a core; and
a selectively permeable coating enclosing said core, said coating comprising a monodisperse polymer.

2. The article of claim 1, wherein said polymer has a molecular weight of from about 1,000 to about 60,000 Da.

3. The article of claim 1, wherein said polymer has a molecular weight of from about 1,000 to about 10,000 Da.

4. The article of claim 1, wherein said polymer has a molecular weight of from about 1,000 to about 4,000 Da.

5. The article of claim 1, wherein said polymer comprises from about 10 to about 300 monomer units.

6. The article of claim 1, wherein said polymer comprises from about 10 to about 40 monomer units.

7. The article of claim 1, wherein said polymer comprises from about 20 to about 30 monomer units.

8. The article of claim 1, wherein said polymer is selected from the group consisting of homopolymers and heteropolymers.

9. The article of claim 1, wherein said polymer is selected from the group consisting of homopolyamino acids, heteropolyamino acids, homooligonucleotides and heterooligonucleotides.

10. The article of claim 1, wherein said polymer comprises amino acids selected from the group consisting of D-amino acids, L-amino acids and combinations thereof.

11. The article of claim 1, wherein said polymer comprises a polyamino acid having a length of from about 10 to about 300 amino acids.

12. The article of claim 1, wherein said polymer comprises a polyamino acid having a length of from about 10 to about 30 amino acids.

13. The article of claim 1, wherein said polymer comprises a polyamino acid.

14. The article of claim 1, wherein said polymer comprises a polylysine.

15. The article of claim 1, wherein said core comprises a hydrogel.

16. The article of claim 1, wherein said core comprises a gel selected from the group consisting of alginate, agar, collagen, chitosan, gelatin, and combinations thereof.

17. The article of claim 1, wherein said core comprises a gel selected from the group consisting of polyacrylamide, polyacrylate, polymethacrylate, and combinations thereof.

18. The article of claim 1, wherein said core comprises an aqueous solution.

19. The article of claim 1, further comprising a second selectively permeable coating on said first coating, said second coating comprising a monodisperse polymer.

20. The article of claim 19, wherein said monodisperse polymer of said second coating is different from the monodisperse polymer of said first coating.

21. The article of claim 1, wherein said coating further comprises a second monodisperse polymer.

22. A device comprising the article of claim 1, wherein said core further comprises a source of a therapeutic substance.

23. The device of claim 22, wherein said therapeutic substance comprises a cell.

24. The device of claim 23, wherein said cell is selected from the group consisting of primary tissue cells, cultured cell lines, genetically engineered cells, stem cells, and combinations thereof.

25. The device of claim 22, wherein said therapeutic substance comprises a drug.

26. The device of claim 22, wherein said therapeutic substance comprises a component capable of a producing a drug.

27. The device of claim 22, wherein said therapeutic substance comprises an islet.

28. The device of claim 22, further comprising a second selectively permeable coating on said first selectively permeable coating, said second selectively permeable coating comprising a monodisperse polymer.

29. The device of claim 28, wherein said second monodisperse polymer differs from said first monodisperse polymer.

30. The device of claim 22, further comprising a gel matrix, said article being disposed in said gel matrix.

31. The device of claim 30, further comprising a second selectively permeable polymer coating enclosing said gel matrix.

32. The device of claim 31, wherein said selectively permeable polymer coating comprises a monodisperse polymer.

33. The device of claim 31, further comprising a second gel matrix surrounding said second selectively permeable polymer coating.

34. The device of claim 30, wherein said gel matrix is biocompatible.

35. The device of claim 31, wherein said second selectively permeable polymer coating is immunoisolating.

36. The device of claim 22, further comprising a gel matrix and a plurality of said articles disposed in said gel matrix.

37. The device of claim 36, further comprising a second selectively permeable polymer coating enclosing said gel matrix.

38. The device of claim 37, wherein said second selectively permeable polymer coating comprises a monodisperse polymer.

39. A device comprising
the article of claim 1, and.
an agent for determining an analyte disposed in said core.

40. The device of claim 39, further comprising a gel matrix, said article being disposed in said gel matrix.

41. The device of claim 40, further comprising a second selectively permeable polymer coating enclosing said gel matrix.

42. The device of claim 41, further comprising a second gel matrix surrounding said second selectively permeable polymer coating.

43. The device of claim 42, wherein said gel matrix is biocompatible.

44. The device of claim 41, wherein said coating is immunoisolating.

45. The device of claim 39, wherein said polymer has a molecular weight of from about 1,000 to about 60,000 Da.

46. The device of claim 39, wherein said polymer has a molecular weight of from about 1,000 to about 10,000 Da.

47. The device of claim 39, wherein said polymer has a molecular weight of from about 1,000 to about 4,000 Da.

48. The device of claim 39, wherein said polymer comprises from about 10 to about 300 subunits.

49. The device of claim 39, wherein said agent is capable of binding to said analyte.

50. The device of claim 39, wherein said agent is capable of reversibly binding to said analyte.

51. The device of claim 39, wherein said agent is capable of reacting with said analyte.

52. The device of claim 39, wherein said agent comprises a reagent selected from the group consisting of energy absorbing reagents, spin resonance reagents, nuclear magnetic resonance reagents, x-ray reagents and combinations thereof.

53. The device of claim 39, wherein said agent comprises a fluorescence reagent.

54. The device of claim 53, wherein said fluorescence reagent exhibits a fluorescence intensity, an emission spectrum, an excitation spectrum, or an excited state lifetime in the presence of said analyte that is different from its fluorescence intensity, emission spectrum, excitation spectrum, or excited state lifetime in the absence of said analyte.

55. The device of claim 53, wherein said fluorescence reagent comprises an energy-accepting donor molecule and an energy-absorbing acceptor molecule, the excited state energy level of the donor overlapping with the excited state energy level of the acceptor.

56. The device of claim 53, wherein said reagent is retained within said device while said analyte is allowed to diffuse into and out of said device.

57. The device of claim 39, wherein said device is implantable in an individual.

58. An in vivo method for determining an analyte in the body fluids of an individual comprising the steps of
a) placing the device of claim 39 in communication with the body fluids of said individual suspected of containing said analyte in such a way that once in place said device does not exit the skin of the individual,
said agent comprising a fluorescence reagent for detecting said analyte that reversibly binds to said analyte,
said fluorescence reagent having a fluorescence intensity, an emission spectrum, an excitation spectrum, or an excited state lifetime in the presence of said analyte that is different from its fluorescence intensity, emission spectrum, excitation spectrum, or excited state lifetime in the absence of said analyte,
said device being configured to retain said fluorescence reagent while allowing said analyte to diffuse into and out of said device;
b) transdermally illuminating said device; and
c) measuring the fluorescence intensity, emission spectrum, excitation spectrum, or excited state lifetime of said fluorescence reagent relative to the fluorescence intensity, emission spectrum, excitation spectrum, or excited state lifetime of said fluorescence reagent in the absence of said analyte; and
d) correlating the change in fluorescence intensity, emission spectrum, excitation spectrum, or excited state lifetime of said fluorescence reagent with the presence or amount of said analyte in said individual.

59. The method of claim 58 wherein said analyte is a carbohydrate.

60. The method of claim 58 wherein said carbohydrate is glucose or a derivative thereof.

61. An in vivo method for determining an analyte in the body fluids of an individual comprising the steps of
a) placing the device of claim 39 in communication with the body fluids of said individual suspected of containing said analyte,
said agent comprising a fluorescence reagent for detecting said analyte that reversibly binds to said analyte,
said device being configured to retain said fluorescence reagent while allowing analyte to diffuse into and out of said device,
said fluorescence reagent comprising an energy-absorbing donor molecule and an energy-absorbing acceptor molecule, the excited state energy level of the donor overlapping with the excited state energy level of the acceptor;
b) transdermally illuminating said device so as to
i) excite the donor or
ii) excite both the donor and acceptor; and
c) measuring the fluorescence from said fluorescence reagent associated with the presence of said analyte in said individual by determining the extent to which non-radiative fluorescence resonance energy transfer occurs between the donor and the acceptor upon binding,
said non-radiative fluorescence resonance energy transfer being determined by measuring
i) the ratio of the fluorescence signal at two emission wavelengths, one of which is due to donor emission and the other of which is due to acceptor emission, when only the donor is excited,
ii) the ratio of the fluorescence signal due to the acceptor following donor excitation and the fluorescence signal due to the acceptor following acceptor excitation,
iii) a change in donor lifetime,
iv) quenching of donor fluorescence, or
v) an enhancement of acceptor fluorescence intensity; and
d) correlating said non-radiative fluorescence resonance energy transfer with the presence or amount of said analyte in said individual.

62. A method of making an article comprising:
applying a selectively permeable monodisperse polymer coating on a core.
